(19) United States
(12) Patent Application Publication
(10) Pub. No.: US 2025/0090212 A1
(43) Pub. Date: Mar. 20, 2025

(54) SYSTEMS AND METHODS FOR TREATING TISSUE WITH A DEPLOYABLE ELECTRODE ARRAY

(71) Applicant: Intuitive Surgical Operations, Inc., Sunnyvale, CA (US)

(72) Inventors: Jonathan COPE, Los Gatos, CA (US); Ryan C. ABBOTT, San Jose, CA (US); Kenneth AYCOCK, Pleasanton, CA (US); William CAMISA, Santa Clara, CA (US); David JOHNSON, Aptos, CA (US); Miles JOSEPH, Sunnyvale, CA (US); Chris JULIAN, Los Gatos, CA (US); Caleb RINGKOB, Menlo Park, CA (US); Zhaoyi ZENG, Fremont, CA (US)

(73) Assignee: Intuitive Surgical Operations, Inc., Sunnyvale, CA (US)

(21) Appl. No.: 18/884,892

(22) Filed: Sep. 13, 2024

Related U.S. Application Data
(60) Provisional application No. 63/583,132, filed on Sep. 15, 2023.

(51) Int. Cl. A61B 18/00; A61B 34/37

(52) U.S. Cl. CPC A61B 18/00; A61B 2018/00107; A61B 2018/0016; A61B 2018/00178; A61B 2018/00214; A61B 2018/00577; A61B 34/37

(57) ABSTRACT

Apparatuses, systems, and methods are disclosed for providing controlled delivery of energy treatment to a tissue site. The systems, apparatuses, and methods may include medical instrument designs with features for retaining a flexible substrate with an electrode array in position against an expandable member during delivery to the tissue site, treatment, and retraction from the tissue site. The medical instrument further includes features for retaining the flexible substrate in a compact, stowed configuration during delivery and for retracting the flexible substrate post-deployment to facilitate retraction of the medical instrument after completion of treatment.

### CROSS-REFERENCE TO RELATED APPLICATION

This application claims priority benefit under 35 U.S.C. § 119(e) of U.S. Provisional Patent Application No. 63/583,132 filed Sep. 15, 2023, the disclosure of which is hereby incorporated by reference in its entirety.

### TECHNICAL FIELD

The present disclosure relates generally to the field of tissue treatment. In particular, the disclosure relates to devices and methods for use with a flexible substrate having one or more electrodes designed for ablating cells to treat targeted tissues.

### BACKGROUND

Tissues in the body may be treated in surgical or medical procedures using resection and removal. While such procedures may be necessary to treat more extensive damage or resolve various medical conditions, minimally invasive procedures may be used as an alternative in some situations. Such procedures, where applicable, may be preferable to reduce overall patient risk, damage to surrounding tissue, recovery time, discomfort, and potentially harmful side effects as compared to more invasive, surgical procedures. In minimally invasive tissue ablation, targeted tissues may be treated inside the body (e.g., in situ) using procedures that do not involve resection or may require minimal resection. Some examples of minimally invasive tissue ablation techniques include electrolytic ablation, cryosurgery, chemical ablation (e.g., alcohol injection), thermal ablation (e.g., radiofrequency, microwave), and hydrothermal ablation. The primary aim of these ablation procedures is to destroy abnormal tissue in a targeted region and encourage the regrowth of healthy tissue.

Another minimally invasive treatment technique involves electroporation of targeted tissues by localized application of an electrical field to increase permeability of cell membranes, which may then allow drugs or other chemicals to be introduced into the cells for treatment or may be used for ablation via a process known as irreversible electroporation. Electroporation may also be used in combination with electrolysis as a method of tissue ablation, by a process also known as electrolytic electroporation, electroporation-electrolysis, or E2.

Tissue ablation is a therapeutic procedure used to treat damage to various tissues, such as those of the gastrointestinal tract, the lungs, the heart, the uterus, endometrial lining, ovaries, ureters, prostate, skin, muscle, and other tissues. Tissue ablation has potential as a minimally invasive therapeutic procedure for targeted treatment for cancer and other ablative applications. By way of example, damage to intestinal tissue may arise from several sources but is commonly associated with chronic metabolic disorders such as diabetes. For patients with diabetes, damage to gastrointestinal tissues (particularly the duodenum) may create issues with insulin resistance and/or impair the ability for the body to process glucose. To help improve patient management of the disorder, duodenal mucosal resurfacing or regeneration (DMR) has been proposed to resurface the patient's intestinal lining and help regenerate healthy lining to improve nutrient absorption at the duodenum. Improved health of the intestinal lining helps correct absorption issues and may yield a better therapeutic response to insulin for the patient, which in turn may allow some patients to replace more aggressive insulin therapies (e.g., injections) with oral medications to help manage the condition.

Generally, DMR is a procedure that involves introducing a catheter (or other suitable medical instrument) into the duodenum, typically under endoscopic guidance, and ablating the inner lining of the duodenum. However, various ablation techniques and ablation instrument designs may not be suitable for endoluminal ablation or may be challenging to control ablation to desired depths via application from within the duodenum lumen.

The inventors have identified a need for an improved medical instrument for use with a flexible substrate having one or more electrodes to ablate cells for treating targeted tissues. Some aspects of the improved medical instrument include a flexible substrate designed for minimizing trauma to surrounding tissues during instrument delivery for treatment and retraction after treatment. Other aspects of the improved medical instrument include features for retaining the flexible substrate in position and resisting dislodgement during delivery and retraction. Still other aspects of the improved medical instrument include features for retaining the flexible substrate in a compact, stowed configuration during treatment, and for controlling retraction of the flexible substrate following treatment to facilitate removal of the medical instrument. Additional aspects and advantages of these and other systems will be apparent from the following detailed description of example embodiments, which proceed with reference to the accompanying drawings.

### SUMMARY

Various examples of systems for treating tissue are described herein. An example system may include a flexible substrate having a header portion and a footer portion, and further include a first peripheral side portion and a second peripheral side portion each spanning between the header portion and the footer portion. The flexible substrate further includes a first arm segment extending outwardly from the header portion and a second arm segment extending outwardly from the footer portion. An electrode array is disposed on the flexible substrate, wherein at least a portion of the electrode array extends onto one of the first arm segment or the second arm segment.

In some example systems, the first arm segment and the second arm segment are aligned relative to one another along a first axis extending across the flexible substrate from the header portion to the footer portion. In some example systems, the first arm segment and the second arm segment are aligned relative to one another along one of the first peripheral side portion or the second peripheral side portion of the flexible substrate. In some example systems, the first arm segment is substantially orthogonal to the header portion and the second arm segment is substantially orthogonal to the footer portion. In some example systems, one of the first arm segment or the second arm segment is free of the electrode array. In some example systems, the first arm segment and the second arm segment are each formed as integral portions of the flexible substrate.

In some example systems, the flexible substrate may include one or more apertures formed thereon, wherein the one or more apertures are disposed between portions of the electrode array. In some example systems, the electrode array comprises a first trace element and a second trace element offset from one another on the flexible substrate, and wherein at least some of the one or more apertures are disposed on the flexible substrate between the first trace element and the second trace element.

In some example systems, the flexible substrate further includes a top surface and an opposite bottom surface, wherein the first peripheral side portion of the flexible substrate includes a first edge surface spanning from the top surface to the bottom surface, and wherein the second peripheral side portion of the flexible substrate includes a second edge surface spanning from the top surface to the bottom surface, the system further comprising a protective coating layer covering at least one of the first edge surface or the second edge surface of the flexible substrate. In some example systems, a protective coating layer covers at least a portion of the top surface of the flexible substrate and may also cover at least a portion of the electrode array disposed on the top surface of the flexible substrate. In some example systems, the protective coating layer covers outer edge surfaces of the flexible substrate.

In some example systems, a length of the first peripheral side portion of the flexible substrate is different than a length of the second peripheral side portion of the flexible substrate. In some example systems, the length of the first peripheral side portion of the flexible substrate is greater than the length of the second peripheral side portion of the flexible substrate. In other example systems, the length of the first peripheral side portion of the flexible substrate is less than the length of the second peripheral side portion of the flexible substrate.

Another example system may include an elongate member and an expandable member coupled to the elongate member, wherein the expandable member is radially expandable relative to the elongate member. The system may further include a flexible substrate including an electrode array disposed thereon, the flexible substrate having a header portion and a footer portion, and a first peripheral side portion and a second peripheral side portion each spanning between the header portion and the footer portion, the flexible substrate including a first arm segment extending outwardly from the header portion and a second arm segment extending outwardly from the footer portion, wherein the flexible substrate surrounds at least a portion of the expandable member when in a stowed position with one of the first arm segment or the second arm segment of the flexible substrate coupled to the expandable member to retain the flexible substrate against the expandable member in the stowed position, and wherein the flexible substrate is expandable from the stowed position to a deployed position in response to radial expansion of the expandable member. In some example systems, the one of the first arm segment or the second arm segment is coupled to the expandable member when the flexible substrate is in the deployed position.

In some example systems, the expandable member is coupled to a distal portion of the elongate member. In some example systems, at least a portion of the elongate member extends distally of the expandable member.

In some example systems, one or more electrical wires are coupled to the electrode array and are configured to power the electrode array, wherein the one or more electrical wires retain the flexible substrate in position along a proximal portion of the elongate member.

In some example systems, the elongate member includes a distal portion and a proximal portion, and wherein the expandable member is coupled to the elongate member between the distal portion and the proximal portion. In some example systems, the one of the first arm segment or the second arm segment of the flexible substrate is coupled to the expandable member adjacent the distal portion of the elongate member to retain the flexible substrate against the expandable member. In some example systems, the other of the first arm segment or the second arm segment of the flexible substrate is coupled to one or more electrical wires configured to power the electrode array, the one or more electrical wires retaining the flexible substrate against the expandable member adjacent the proximal portion of the elongate member.

In some example systems, the flexible substrate further includes a leading edge defined along one of the header portion or the footer portion of the flexible substrate corresponding with the one of the first arm segment or the second arm segment coupling the flexible substrate to the expandable member when the flexible substrate is in the stowed position, the system further including a first cushion adjacent the leading edge of the flexible substrate to protect against tissue trauma during system delivery. In some example systems, the first cushion contacts the leading edge of the flexible substrate. In some example systems, the flexible substrate further includes a trailing edge defined along the other of the header portion or the footer portion of the flexible substrate, the system further including a second cushion adjacent the trailing edge of the flexible substrate to protect against tissue trauma during system removal. In some example systems, the second cushion contacts the trailing edge of the flexible substrate.

In some example systems, the flexible substrate further includes a trailing edge defined along the other of the header portion or the footer portion of the flexible substrate, wherein the expandable member is partially expanded to form a second cushion adjacent the trailing edge of the flexible substrate to protect against tissue trauma during system removal. In some example systems, the second cushion contacts the trailing edge of the flexible substrate.

Another example system includes an elongate member and an expandable member coupled to the elongate member, wherein the expandable member is radially expandable relative to the elongate member. The system may further include a flexible substrate including an electrode array disposed thereon, wherein the flexible substrate is movable from a stowed position wherein the flexible substrate surrounds at least a portion of the expandable member and has a first outer diameter, to a deployed position wherein the flexible substrate is expanded to a second outer diameter larger than the first outer diameter in response to radial expansion of the expandable member. An adjustment member is coupled to at least one of the flexible substrate or the expandable member, wherein the adjustment member is adjustable to retain the flexible substrate in the stowed position.

In some example systems, the adjustment member is further adjustable to control movement of the flexible substrate from the stowed position to the deployed position after an initial adjustment to retain the flexible substrate in the stowed position. In some example systems, the adjustment member is further adjustable to drive the flexible substrate from the deployed position to the stowed position.

In some example systems, the flexible substrate further includes one or more apertures formed thereon, and wherein the adjustment member is coupled to the flexible substrate through at least some of the one or more apertures. In some example systems, the adjustment member is a cable or line that is weaved through at least some of the one or more apertures formed on the flexible substrate to couple the adjustment member to the flexible substrate.

In some example systems, the adjustment member is releasable from the flexible substrate to facilitate movement of the flexible substrate from the stowed position to the deployed position.

In some example systems, the adjustment member limits expansion of the flexible substrate in the deployed position to a predetermined outer diameter. In some example systems, the flexible substrate further includes one or more apertures formed thereon, and wherein the adjustment member is coupled to the flexible substrate through at least some of the one or more apertures, the adjustment member contacting the flexible substrate to limit expansion to the predetermined outer diameter.

In some example systems, the flexible substrate is coupled to the expandable member, and wherein the adjustment member is coupled to the expandable member, wherein the adjustment member adjusts the expandable member to retain the flexible substrate in the stowed position.

In some example systems, the adjustment member comprises a tensioned element, such as a line or a cable, wherein the tensioned element adjusts the flexible substrate to retain the flexible substrate in the stowed position in response to tension applied to the tensioned element. In some example systems, the tensioned element itself and/or tension of the tensioned element may be releasable, such as via cutting of the tensioned element, to facilitate adjustment of the flexible substrate from the stowed position to the deployed position. In some example systems, the tensioned element drives the flexible substrate from the deployed position to the stowed position in response to tension applied to the tensioned element.

In some example systems, the flexible substrate further includes one or more apertures formed thereon, and wherein the adjustment member is a tensioned element coupled to the flexible substrate through at least some of the one or more apertures. In some example systems, the tensioned element is a cable or line that is weaved through at least some of the one or more apertures formed on the flexible substrate to couple the tensioned element to the flexible substrate.

In some example systems, the flexible substrate further includes one or more apertures formed thereon, and the adjustment member is a tensioned element coupled to the flexible substrate through at least some of the one or more apertures, the tensioned element contacting the flexible substrate to limit expansion to the predetermined outer diameter.

Another example system for tissue treatment includes a flexible substrate having a proximal end and a distal end, and a first peripheral side portion and a second peripheral side portion each spanning between the proximal end and the distal end. The system further includes an electrode array disposed on the flexible substrate, wherein the electrode array includes a plurality of spaced apart traces with a first trace being closest to the proximal end of the flexible substrate and a second trace being closest to the distal end of the flexible substrate. The flexible substrate includes a buffer region without an electrode trace, the buffer region disposed between at least one of: a) a distance between the first trace and the proximal end of the flexible substrate; or b) a distance between the second trace and the distal end of the flexible substrate, and wherein a length of the buffer region is equal to or greater than a spacing between adjacent traces of the electrode array. In some example systems, the buffer region is between both a) the distance between the first trace and the proximal end of the flexible substrate; and b) the distance between the second trace and the distal end of the flexible substrate. In some example systems, a length of the buffer region between the first trace and the proximal end of the flexible substrate is greater than a length of the buffer region between the second trace and the distal end of the flexible substrate. In other example systems, a length of the buffer region between the first trace and the proximal end of the flexible substrate is smaller than a length of the buffer region between the second trace and the distal end of the flexible substrate. In some example systems, the buffer region ranges from between 3 mm to 7 mm in length.

It should be understood that the foregoing summary provides various examples further described herein and is not intended to identify any key or critical aspects of the disclosed or claimed subject matter. Further, aspects of the example systems summarized above may be combined in any suitable manner without departing from the principles of the disclosed or claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are therefore not to be considered limiting in scope, the disclosure will be described with additional specificity and detail through use of the accompanying drawings, in which:

FIG. 1 is a schematic illustration of a system for electroporation and/or electrolysis in accordance with examples described herein.

FIG. 2 is a schematic illustration of a robotically-assisted manipulator system in accordance with examples described herein.

FIG. 3A is a schematic diagram of an instrument system in accordance with examples described herein.

FIG. 3B illustrates a distal portion of the instrument system of FIG. 3A with an extended example of an instrument in accordance with examples described herein.

FIG. 4A illustrates a catheter in a sheathed configuration, the catheter including an electrode array for delivery to a target region in accordance with examples described herein.

FIG. 4B illustrates the catheter of FIG. 4A in an unsheathed configuration in accordance with examples described herein.

FIG. 5 illustrates another catheter with an electrode array for delivery to a target region in accordance with examples described herein.

FIG. 6 illustrates a standalone flexible substrate with an electrode array of the catheter of FIG. 5.

FIG. 7A illustrates a flexible substrate including a protective coating layer along edge portions of the flexible substrate in accordance with examples described herein.

FIG. 7B illustrates another flexible substrate including a protective coating layer along edge portions of the flexible substrate in accordance with examples described herein.

FIG. 8 illustrates an example catheter with the flexible substrate of FIG. 7A.

FIG. 9 illustrates a catheter with an electrode array having distal and proximal cushions for minimizing trauma during delivery to a target region in accordance with examples described herein.

FIG. 10 illustrates a catheter with an electrode array with distal and proximal portions of an expandable member partially expanded for minimizing trauma during delivery to a target region in accordance with examples described herein.

FIG. 11A illustrates a catheter with an adjustment member operable for retaining the flexible substrate in a stowed position during delivery to a target region in accordance with examples described herein.

FIG. 11B illustrates a schematic view of a catheter with an adjustment member in accordance with examples described herein.

FIG. 11C illustrates another schematic view of a catheter with an adjustment member in accordance with examples described herein.

FIG. 11D illustrates a schematic view of a flexible substrate with an adjustment member for retaining the flexible substrate in a stowed position during delivery to a target region in accordance with examples described herein.

FIG. 11E illustrates another schematic view of a flexible substrate with an adjustment member for retaining the flexible substrate in a stowed position during delivery to a target region in accordance with examples described herein.

FIG. 11F illustrates a flexible substrate including one or more adjustment members along edge portions of the flexible substrate for retaining the flexible substrate against an expandable member in accordance with examples described herein.

FIG. 12 is a schematic end view of the flexible substrate and the adjustment member of FIG. 11A.

FIG. 13 illustrates a catheter with a keyed sheath operable to mate with the flexible substrate for retracting the flexible substrate to facilitate retraction of the catheter upon completion of treatment in accordance with examples described herein.

FIG. 14 is a schematic illustration of a segmented flexible substrate and electrode array system for use with a catheter in accordance with examples described herein.

FIG. 15 is a schematic illustration of a catheter in accordance with examples described here.

FIG. 16A is a schematic illustration of a flexible substrate and electrode array system around an expandable member in accordance with examples described herein.

FIG. 16B is a schematic illustration of the flexible substrate of FIG. 16A illustrating a partially shifted electrode array system upon expansion of the expandable member in accordance with examples described herein.

FIG. 16C is another schematic illustration of a flexible substrate with an electrode array system for use with a catheter in accordance with examples described herein.

FIG. 16D is yet another schematic illustration of a flexible substrate with an electrode array system for use with a catheter in accordance with examples described herein.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of the claimed subject matter to these particular embodiments. In other instances, well-known materials, components, processes, controller components, software, and/or anatomy have not been described or shown in detail in order to avoid unnecessarily obscuring the embodiments.

This disclosure is directed to apparatuses and systems for providing controlled delivery of one or more electrodes (e.g., one or more pairs of electrodes) to a targeted tissue site for treatment deployed via a flexible substrate. Generally, a method for minimally invasive regenerative surgery is disclosed which includes subjecting a target area in living tissue to ablation delivered via the one or more pairs of electrodes. In some examples, the ablation energy may be in the form of a combination of one or more electric fields and electrolysis. However, it should be appreciated that the example systems and methods described herein may be utilized for the deployment of flexible substrates carrying electrodes using ablation modalities other than electroporation and/or electrolysis (e.g., radiofrequency ablation).

During an example procedure utilizing electroporation and electrolysis, electrodes are brought into proximity and/or contact with the target tissue, and the electric fields are generated by applying voltages and/or currents between the one or more pairs of electrodes. The electric fields may be generated to have a magnitude to permeabilize cell membranes in a region where ablation is desired. The electric fields may be generated to produce products of electrolysis of a magnitude that, by themselves, do not cause damage to cells or the extracellular matrix located in and around the target treatment region. However, when sufficient products of electrolysis are generated in the region of permeabilized cells, cellular death occurs within the region of the applied electric field without damaging the extracellular matrix or scaffolding to aid in promoting tissue regrowth in the treated tissue region.

In some examples, after applying ablative energy to one target region, the electrodes may be moved to other tissue locations by advancing and/or retracting a delivery catheter and the electrodes carried by the delivery catheter as needed and repeating the process. In this manner, electroporation and electrolysis may be performed at multiple locations in a patient to cover a larger area of tissue. In some examples, multiple sets of electrodes may be positioned at multiple respective tissue sites such that electroporation and electrolysis may be performed at each site in parallel, reducing and/or eliminating a need to repeat the procedure as a catheter is moved through the patient.

Following ablation using electroporation and electrolysis, tissue regeneration may occur at the treatment site, aided by the extracellular matrix left behind. The tissue may regenerate from stem cells introduced to the treatment site via the circulatory system, from stem cells found at the margins of the ablated tissue, and/or from cells or other components otherwise introduced to the treatment site. The stem cells then proliferate along the intact extracellular matrix and into the ablated area to replace the ablated tissue with healthy tissue. While regeneration may be achieved at the treatment site through natural processes, there are other ways to repopulate the extracellular matrix with cells which are different from the original cells at that location. For example, genetically engineered cells may be injected into the treatment site to facilitate regrowth. This process may be useful in instances where the original tissue cells had genetic or other abnormalities. In another example, other cells (e.g., pancreatic islets) may be introduced to the treatment site for implantation and proliferation. In other embodiments, other methods may be used to promote tissue regeneration at the treatment site.

In some embodiments of the present disclosure, the systems and methods disclosed herein may be used to treat tissue of the gastrointestinal tract. In other embodiments, any of a variety of tissue may be treated using the systems and methods described herein. Generally, tissue may be treated where tissue regeneration is desirable or where it is desirable to replace one type of cells with another. Examples include intestine, duodenum, stomach, bladder, uterus, endometrial lining, ovaries, colon, rectum, sinuses, ducts, ureters, prostate, skin, muscle, nerve, diaphragm, momentum, kidney, follicles, brain, lymphatic vessels, breast, esophagus, lung, liver, kidney, lymph nodes, lymph node basins and/or heart. Replacement of one type of tissue with another may be in fibrotic areas where it is desired to replace fibrotic cells with stem cells that can remodulate the area or when pancreatic islets are injected in part of the liver to generate new sources of insulin. Other tissue may be treated in other examples. The following provides additional details relating to example processes of electroporation and/or electrolysis for tissue treatment in accordance with some embodiments.

As noted above, electroporation may be performed to permeabilize the cell membranes of targeted cells. Reversible electroporation may be used in which the permeabilization may cease after the electric fields are removed. Cells may survive reversible electroporation with the pores within the membrane resealing and returning to homeostasis. In irreversible electroporation, the permeabilization of the cell membrane is permanent leading to cell death. Typically for biological tissues, electric fields lower than about 1500 V/cm to about 200 V/cm are considered to produce reversible electroporation, while electric fields higher than about 1500 V/cm are considered to produce irreversible electroporation. Examples of systems and methods described herein may utilize reversible electroporation to avoid disadvantages of irreversible electroporation, which may include heating and thermal damage, complexity of providing such large electric fields, and muscle contractions which may result from the large electric fields. It is to be understood that, although systems and methods described herein may be designed to utilize reversible electroporation, there may occur localities or incidental areas where conditions are such that irreversible electroporation, or even thermal ablation, may occur in limited parts of the treated tissue.

In some treatment procedures, products of electrolysis may be applied to permeabilized cells to cause cell death of the permeabilized cells within the applied electrical field while leaving the extracellular matrix of the permeabilized cells intact. The extracellular matrix generally refers to a three-dimensional network of proteins and/or other molecules (e.g., collagen fibers, proteoglycans, and/or proteins such as fibronectin and/or laminin) which provide structure for cells and tissues and may additionally provide signaling for cell growth and development. Extracellular matrices may be used as scaffolds for tissue regeneration and/or engineering. For example, cells may be regenerated, grown, transplanted, or otherwise nurtured, on the extracellular matrix. By retaining the extracellular matrix in regions of otherwise ablated cells described herein, cell regeneration and/or tissue engineering may occur in the region of ablated cells. In some procedures, the extracellular matrix may be transplanted from a region of ablated cells to another region (which may be another region of ablated cells), to promote regeneration and/or tissue engineering in the transplanted region. In some cases, once the extracellular matrix is present, material may be injected into the extracellular matrix to enhance regrowth.

One aspect of the ablation technology that may be applicable to this disclosure accordingly relates to the use of electrolysis in the process of ablation. The process of electrolysis refers to an electrochemical reaction that occurs at the electrode surfaces of electrodes in contact with an ionic conducting media (e.g., an aqueous solution, including a native physiological concentration solution present in the tissue). The electrochemical reaction occurs generally as a result of an electric potential driven transfer between electrons from the electrode and ions or atoms in the solution. The chemical species generated on the electrodes diffuse in the ionic conductive media from the electrodes outward in an electroosmotic diffusion process. In an environment typical of biological matter, these chemical reactions also yield changes in pH, resulting in an acidic region near the anode and a basic region near the cathode as well as the production of chemical species which can be toxic to biological matter. These products and changes in pH diffuse from the electrodes into the biological media. Electrolysis alone (e.g., without cell membrane permeabilization) is a well-known chemical ablation mechanism, and the extent of ablation is a function of the nature of the chemical species and its concentration. Because the products of electrolysis are generated at the electrodes, to ablate large volumes of biological matter, the products of electrolysis must diffuse throughout the targeted volume. Diffusion is a relatively slow process such that electrolytic ablation using ablation from electrolysis alone results in a lengthy procedure. For example, the products of electrolysis and the tissue ablation treatment using electrolysis alone may occur over a period on the order of magnitude of tens of minutes to hours. This is because of the concentration of cytotoxic products of electrolysis that must diffuse throughout the entire target volume to ablate that volume. Additionally, when electrolysis alone is used to ablate tissue, the concentration and exposure time to electrolysis products are such that scarring, ulceration, coagulative necrosis, and/or fibrosis results.

Electrolysis preferentially utilizes inert electrodes that do not participate in the process of electrolysis except as a source or sink of electrons or as catalysts. When participating non-inert electrodes are used in the process, they can generate metal ions that may cause systemic damage to the body, such as excess of iron or even metallic fragments. Electrolysis products may include cytotoxic products. For example, an abundance of protons H⁺ can generate a non-physiological acidic environment diffusing from the anode and an abundance of OH⁻ can generate a non-physiological basic environment which diffuses from the cathode. Permeabilized cells exposed to non-physiological pH can die because the intracellular homeostasis is disrupted, and the normal cell chemical pathways are disrupted. The products of electrolysis can combine and generate cytotoxic chemicals such as hypochlorous acid or hydrochloric acid whose effect may depend in part on the pH in the region where they form. For example, passing a current through a saline solution (NaCl and H₂O) at a pH between 3 and 5 may generate extremely toxic hypochlorous acid (HOCl). Products of electrolysis affect biological matter through chemical reactions in which they are involved, as a function of their concentration and time of exposure. High concentrations and longtime exposure to products of electrolysis affect all the biological molecules in the targeted tissue, an undesirable effect for tissue regeneration applications. However, judiciously delivering products of electrolysis at lower concentrations for shorter periods of time at a level at which they are unable to affect all the biological molecules in a treated volume of tissue and are able to only affect cells whose membrane is temporarily or permanently permeabilized by diffusing into the cell and affecting the intracellular homeostasis can cause cell death without affecting the extracellular matrix. For example, typical methods for tissue ablation by electrolysis utilize the delivery of charge of about 30-100 Coulombs per cm diameter of targeted tissue and typically 40 mA delivered for 90 minutes. In some systems for regenerative electrolytic electroporation ablation, exponentially decaying currents may be used having decaying amplitudes for much shorter times—a peak of 250 mA delivered for less than 2 ms in one example. Accordingly, the current provided for electrolysis may be provided using an exponential decay or other decaying waveform and may have an amplitude of about tens or hundreds of mA. The current may be provided for a time scale of about milliseconds or may be provided in multiple pulses (e.g., multiple waveforms).

Example systems and methods described herein may utilize electrolysis products to cause cell death together with electroporation to permeabilize the cell membrane. The electrolysis products are sufficient to ablate (e.g., cause cell death) permeabilized cells in a relatively short time frame. However, the concentration of electrolysis products and exposure time are insufficient to cause cell death of non-permeabilized cells, thereby minimizing or avoiding the formation of scar tissue, fibrotic tissue, or ulceration in the treated region. Scar tissue and fibrosis is an indication that the extracellular matrix has been affected, and the ability for cells to regenerate and/or tissue engineering to occur in the ablated region may be inhibited. Accordingly, the electrolysis products are used to ablate permeabilized cells only, while leaving the extracellular matrix intact to promote tissue regrowth.

Examples of systems disclosed herein may include electrodes, a power supply, and a controller to apply electrolysis and/or electroporation for treating internal tissue. The controller may control a charge delivered to the electrodes to induce one or more electric fields. For example, the electrodes may be used to generate a current to produce electrolysis products and a voltage difference to produce an electric field that induces electroporation. The duration and magnitude of the charge applied may determine the dose of the electrolytic products and the degree of the permeabilization of cells in the treatment site. Accordingly, a region of cell ablation may be determined by a region in which cells are exposed to the combination of permeabilization and to electrolysis products that cause ablation. The ablation, however, may leave the extracellular matrix intact in the region of ablated cells where the electrical field has been applied. The composition of the electrodes may be chosen in accordance with the desired products produced and electroporation effects.

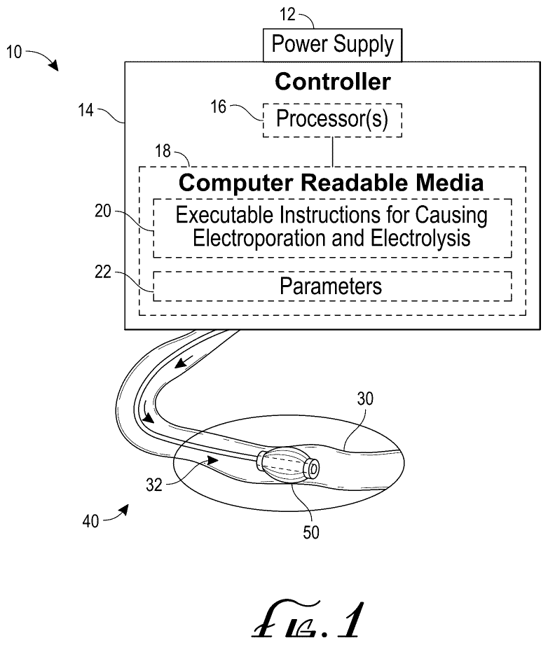

With reference to FIG. 1, the following provides a brief overview of an example system for delivering a catheter with electrodes to a targeted tissue site for treatment via electroporation and/or electrolysis. For clarity, the written description and the associated figures of various example embodiments generally references a catheter for use with the systems and methods described herein. However, it should be understood that other suitable medical instruments (e.g., instruments that may not be specifically classified as catheters) and other suitable energy modalities (e.g., radiofrequency ablation) may be used in conjunction with the disclosed systems and methods without departing from the principles of the disclosed subject matter.

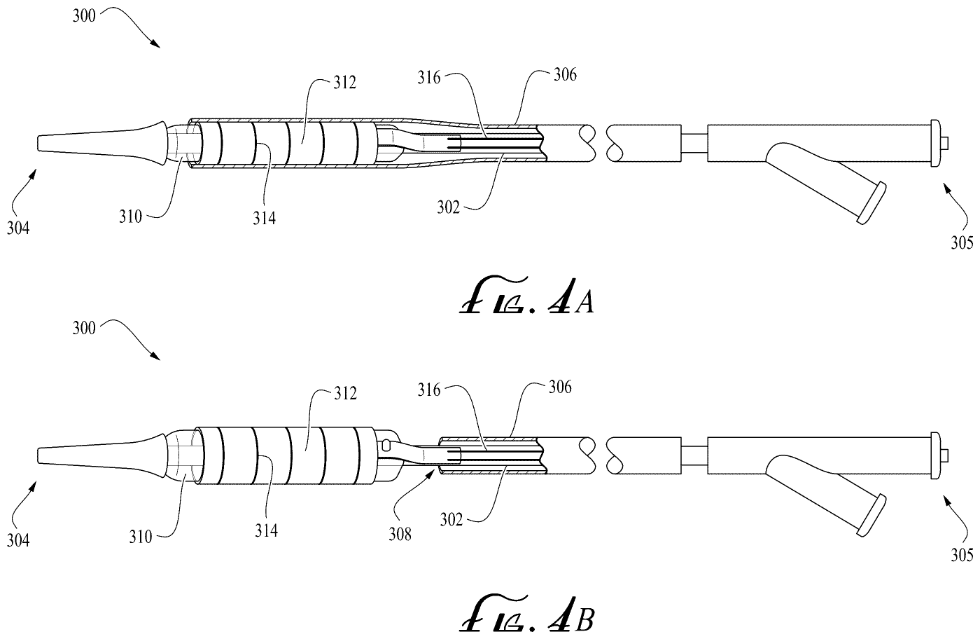

FIG. 1 is a schematic illustration of a system 10 arranged in accordance with example embodiments described herein. Generally, example systems described herein may include a delivery system and a controller. In the example of FIG. 1, the system 10 includes a controller 14 and a catheter 40. Examples of other suitable catheters which may be used with the system 10 are described in further detail below with respect to FIGS. 4A-16D. As further described below, the controller 14 may include a processor 16, computer readable media 18, and other computing system components, such as one or more input devices, output devices, sensors, and/or communication devices in some examples. Additional, fewer, and/or different components may be used in other examples. The computer readable media 18 includes executable instructions for causing electroporation and electrolysis 20 with the catheter 40 and may include stored parameters 22 which may be used in the process for causing electroporation and electrolysis, such as electric field strengths, voltage and/or current levels, waveform shapes, exposure duration parameters, and any other suitable parameters. In the example of FIG. 1, the circle depicted around an end of the catheter 40 may indicate a region of cells that may be permeabilized via an applied electric field.

The controller 14 may be implemented using a computing device. Examples of computing devices include controllers, microcontrollers, computers, servers, medical devices, smart phones, tablets, wearable devices, and the like. The computing device may be handheld and may have other uses as well. The controller 14 may include one or more processors, such as the processor 16. Any kind or number of processors may be present, including one or more central processing unit(s) (CPUs) or graphics processing unit(s) (GPUs) having any number of cores, controllers, microcontrollers, and/or custom circuitry such as one or more application specific integrated circuits (ASICs) and/or field programmable gate arrays (FPGAs).

The controller 14 described herein may include computer readable media 18, such as memory. Any type or kind of memory may be present (e.g., read only memory (ROM), random access memory (RAM), solid state drive (SSD), secure digital card (SD card), and the like). While a single box is depicted as the computer readable media 18 in FIG. 1, any number of computer readable media 18 devices may be present. The computer readable media 18 may be in communication with (e.g., electrically connected to) the processor 16.

As noted above, the computer readable media 18 may store executable instructions for execution by the processor 16, such as executable instructions for causing electroporation and electrolysis 20 with the catheter 40 and may utilize stored parameters 22 for the catheter 40. In this manner, techniques for applying electroporation and electrolysis in tissue may be implemented herein wholly or partially in software.

The executable instructions 20 may include instructions to control a charge delivered to electrodes, such as electrodes 50 of the catheter 40. Accordingly, the controller 14 may induce a voltage difference across the targeted tissue to generate an electric field that causes permeabilization of cells in an area of tissue targeted for treatment. In the illustrated embodiment, the electrodes 50 may be disposed on a flexible substrate (e.g., flexible substrate 312 of FIG. 4A) of the catheter 40 as described in further detail with particular reference to FIGS. 4A and 4B. The electrodes 50 are illustrated in a cavity or lumen 32 formed within tissue 30. Although the catheter 40 is shown disposed within a cavity 32 of tissue 30, the catheter 40 may be on the surface of the tissue 30, inside the tissue 30, and/or proximate to the tissue 30. Moreover, although a catheter 40 is shown for delivering and arranging the electrodes 50 for permeabilization and/or the generation of electrolysis products to treat tissue 30, in other embodiments, other suitable delivery systems may be used.

A controller, such as controller 14 of FIG. 1, may activate one or more selected electrodes 50 to provide an electric field. In some embodiments, the controller 14 may alternate or otherwise select a pattern of activated electrodes 50 (e.g., activating pairs of electrodes in sequence) to shape or deliver a particular electric field. In some examples, fluids or other substances may be injected into, brought into contact with, or otherwise placed in or around the tissue 30 to aid in shaping the electric field generated in the tissue 30. For example, conductive fluids may aid in shaping the field (e.g., by extending the field). In other examples, non-conductive fluids may aid in shaping the field (e.g., by attenuating the field). In some examples, non-conductive fluids or other substances may be injected or otherwise placed in tissue to protect areas where ablation is not desired. The electric field may not penetrate and/or not be carried through the non-conductive fluid, such that the field would not reach tissue where ablation is not desired, or at least be present in insufficient strength to cause permeabilization or other cellular change.

The controller 14 may also be used to induce a current through the tissue 30, such as between electrodes 50, to generate products of electrolysis. The products of electrolysis may cause ablation of the permeabilized cells but are preferably insufficient to destroy the extracellular matrix in the region of the permeabilized cells. The remaining intact extracellular matrix may allow for regeneration of the tissue and tissue engineering as described previously.

In some embodiments, one or more of the electrodes 50 used to apply electroporation may also be used to generate products of electrolysis (in other words, some or all of the electrodes may be used for both electroporation and electrolysis). In other embodiments, a first subset of electrodes 50 used to apply electroporation may be different from a second subset of electrodes 50 used to generate products of electrolysis.

In some embodiments, the power supply 12 is integrated with the controller 14. The power supply 12 may be implemented using any suitable power source, such as one or more AC power sources, DC power sources, batteries, and/or waveform generators. The power supply 12 may supply power to the electrodes 50 to generate a voltage and/or current and, therefore, an electric field and/or electrolysis products in the tissue 30. In some examples, the power supply 12 may be implemented using a signal generator, such as an exponential decay wave generator (by way of example, a Harvard Apparatus BTX 630), however, this disclosure is not limited thereto or thereby.

The controller 14 may control the timing, strength, and duration of electric fields and/or electrolysis products provided via the catheter 40. The controller 14 may, for example, be programmed to provide an electronic signal to the catheter 40, via the power supply 12, where the electronic signal may be indicative of a dose of treatment, for example, a dose of electrolysis products and/or permeability level of cell. The electronic signal may control the timing and magnitude of the generated electric field, which may allow a user to customize treatment of the tissue 30 as desired. In some embodiments, the controller 14 may include such a program, or include one or more processing devices (e.g., processors), coupled to the computer readable media 18 encoded with executable instructions for electrolysis and permeabilization 20. Although shown as a separate component coupled to the catheter 40 in FIG. 1, in some embodiments, the controller 14 may be integrated as part of the catheter 40. In other embodiments, the controller 14 may include programmable circuitry coupled to the catheter 40 via a wired or wireless connection.

As noted previously, the system 10 may include any suitable parameters 22 for controlling various aspects of the electroporation and electrolysis processes, such as electric field strengths, voltage levels, current levels, waveform shapes, exposure duration parameters, and any other suitable parameters. The parameters 22 may be stored in computer readable media 18 or in other suitable databases in communication with the controller 14. In some embodiments, the controller 14 may be used to calculate the parameters 22, or the controller 14 may be in communication with another system operable to calculate the parameters 22.

In some example embodiments, the parameters 22 for a specific treatment protocol may be determined based on measurements taken in the tissue of interest or from a different sample of similar tissue. For example, measurements may be taken at various voltage levels with particular electrode configurations, and a target voltage level, current, pulse pattern, time constant, and other factors may be identified which cause reversible electroporation and the delivery of electrolysis products to result in cell death of the permeabilized cells as desired.

In some embodiments, examples of parameters 22 which may be used include a delivery of between 1 and 10 voltage pulses between 100 V and 1000 V. Those pulses may be delivered in a system having a capacitance between 50 μF and 100 μF, and a resistance of between 15-20 ohms. A quantity of electrolysis products generated may be related to the delivered charge in Coulombs. There are several ways to calculate the delivered charge. For example, the stored electric charge in a capacitor Q (in coulombs, abbreviated C) is equal to the product of the capacitance C (in Farads, abbreviated F) of the capacitor, and the voltage V (in volts, abbreviated V) across its terminals. That is, Q=C·V. Also, current I (in amps, abbreviated A) multiplied by time t=charge. I*t=Q. By defining the capacitance and the voltage across the capacitance, the charge may be defined, and accordingly the electrolysis performance determined. When a capacitor is discharged, it generates current and the current multiplied by time must be equal to the charge in the capacitor. When a capacitor is being discharged the current is not constant—it decays exponentially. Therefore, the time measure is given as the exponential decay time constant. The capacitance which controls the time constant is generally obtained from capacitors incorporated in the power supply, such as power supply 12 of FIG. 1.

In some embodiments, the time constant (e.g., exponential decay time constant of the capacitive discharge) may be between 1.7 milliseconds (ms) and 1.8 ms seconds. In other embodiments, the time constant may range from 50 microseconds (μs) to 3 ms. Generally, the lower limit of the time constant is related to a time sufficient to ensure electrolytic species (e.g., products of electrolysis) permeate the targeted area of permeabilized cells. The upper limit of the time constant is generally related to the production of electrolytic species (e.g., products of electrolysis) that can cause ablation on their own. Accordingly, electrolysis is generally targeted for an amount of time sufficient to allow diffusion of electrolysis products through a region of permeabilized cells. However, the amount of time electrolysis is provided should be limited to ensure the process does not result in ablation of non-permeabilized cells or otherwise damages the extracellular matrix in the region of the ablated cells.

In some embodiments, the generated electric field ranges between 100 V/cm and 3500 V/cm. In other embodiments, the electric field may be between 100 V/cm and 1500 V/m or between 200 V/cm and 850 V/cm. In still other embodiments, the electric field may be less than 1400 V/cm in some examples, less than 1300 V/cm in some examples, less than 1000 V/cm in some examples, less than 800 V/cm in some examples, or less than 600 V/cm in some examples.

The system 10 may further include one or more sensors (not shown) for measurement of pH, temperature, electric field strength, tissue electrical resistivity or impedance, and/or other suitable properties of the tissue 30 for optimizing treatment. For example, in one embodiment, a pH sensor may be incorporated with the system 10. The pH sensor may be arranged in any one of several configurations, such as coupled to the catheter 40 adjacent the electrodes 50 to detect a pH value near the electrodes 50. In another embodiment, a pH sensor may be provided at an outer edge of a targeted region of tissue. In either configuration, the pH sensor may be in communication with the controller 14, where the controller 14 may utilize one or more received pH values as an indication of tissue ablation and/or to monitor the occurrence of tissue damage based on detected pH levels at the treatment site and surrounding regions of tissue. In some embodiments, the controller 14 may adjust the voltage, current, and/or electric field applied to the tissue responsive to the detected pH levels. For example, if pH values for tissue located outside the treatment site are at or exceed a threshold for tissue damage, the controller 14 may reduce a magnitude of electric field, a duration between pulses, or cease application of the electric field. Similarly, if pH values for tissue within a targeted region are at or exceed a threshold for tissue ablation, the controller 14 may cease application of current through electrodes immediately and/or after a desired elapsed electrolysis time to cease the electrolysis process.

In some embodiments, a resistivity meter may be used to determine a resistance of the target tissue. For example, the controller 14 and/or power supply 12 of FIG. 1 may provide an impedance measurement. The impedance measurement may determine a resistivity of the tissue 30 contacted by electrodes 50 of the system 10. For example, the controller 14 and/or power supply 12 may provide a nominal amount of current, such as DC current, through the tissue 30 and receive a resistivity measurement and/or calculate resistivity of the tissue 30. In some examples, an applied voltage, current, and/or electric field may be selected, determined, and/or allowed based on a measured resistance of the tissue 30. In some examples, a number of pulses of applied voltage may be selected, determined, and/or otherwise used based on a measured resistance of the tissue.

In some embodiments, a sensor, such as a Gauss meter and/or Tesla meter, for detecting and/or determining electric field strength may also be used. In such embodiments, the sensor may be in operable communication with the controller 14 for ensuring that the electric field strength remains at a target level for a desired treatment protocol.

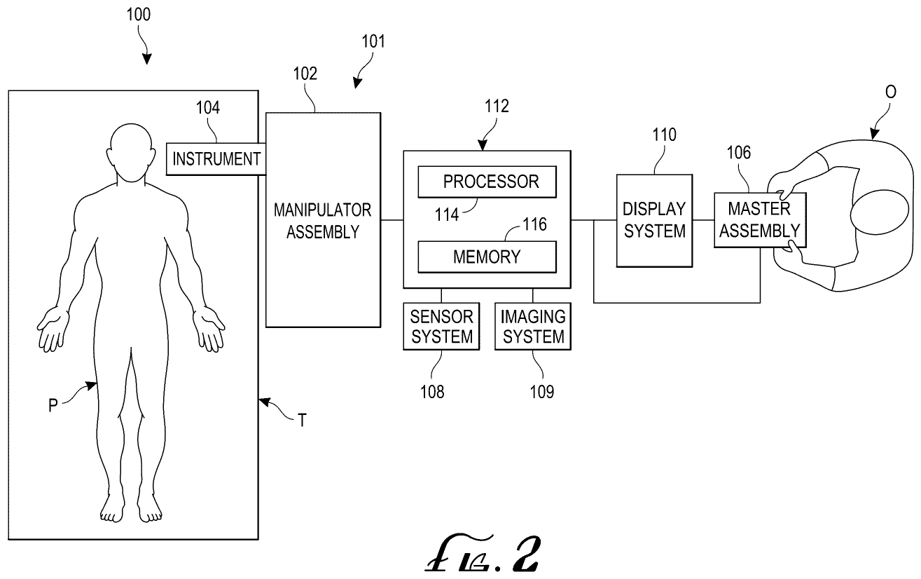

In some embodiments, the ablation instrument for delivering electrolytic electroporation (e.g., catheter 40 of FIG. 1) may be delivered via a computer-assisted, teleoperational manipulator system, sometimes referred to as a robotically assisted system or a robotic system. The manipulator system comprises one or more manipulators that can be operated with the assistance of an electronic controller (e.g., computer) to move and control functions of one or more instruments when coupled to the manipulators. FIG. 2 illustrates one example embodiment of a robotically-assisted manipulator system 100 for use with the systems and methods described herein. The manipulator system can be used, for example, in surgical, diagnostic, therapeutic, biopsy, or non-medical procedures.

With reference to FIG. 2, a robotically-assisted manipulator system 100 may include one or more manipulator assemblies 102 for operating one or more medical instrument systems 104 in performing various procedures on a patient P positioned on a table T in a medical environment 101. For example, the manipulator assembly 102 may drive catheter or end effector motion, may apply treatment to target tissue, and/or may manipulate control members. The manipulator assembly 102 may be teleoperated, non-teleoperated, or a hybrid teleoperated and non-teleoperated assembly with select degrees of freedom of motion that can be motorized and/or teleoperated and select degrees of freedom of motion that may be non-motorized and/or non-teleoperated. An operator input system 106, which may be inside or outside of the medical environment 101, generally includes one or more control devices for controlling manipulator assembly 102. The manipulator assembly 102 supports a medical instrument system 104 and may optionally include a plurality of actuators or motors that drive inputs on the medical instrument system 104 in response to commands from a control system 112. The actuators may optionally include drive systems that when coupled to the medical instrument system 104 advance the medical instrument system 104 into a naturally or surgically created anatomic orifice. Other drive systems can move the distal end of the medical instrument system 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 102 may support various other systems for irrigation, treatment, or other purposes. Such systems may include fluid systems (including, for example, reservoirs, heating/cooling elements, pumps, and valves), generators, lasers, interrogators, and ablation components.

The robotically-assisted manipulator system 100 also includes a display system 110 for displaying an image or representation of the surgical site and the medical instrument system 104 generated by an imaging system 109 which may include an imaging system, such as an endoscopic imaging system. The display system 110 and the operator input system 106 may be oriented so an operator O can control the medical instrument system 104 and the operator input system 106 with the perception of telepresence. A graphical user interface may be displayable on the display system 110 and/or a display system of an independent planning workstation.

In some examples, the endoscopic imaging system components of the imaging system 109 may be integrally or removably coupled to the medical instrument system 104. However, in some examples, a separate imaging device, such as an endoscope, attached to a separate manipulator assembly may be used with the medical instrument system 104 to image the surgical site. The endoscopic imaging system 109 may be implemented as hardware, firmware, software, or a combination thereof which interact with or are otherwise executed by one or more computer processors, which may include the processors of the control system 112.

The robotically-assisted manipulator system 100 may also include a sensor system 108. The sensor system 108 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 may also include temperature, pressure, force, or contact sensors or the like.

The robotically-assisted manipulator system 100 may also include a control system 112. The control system 112 includes at least one memory 116 and at least one computer processor 114 for effecting control between the medical instrument system 104, the operator input system 106, the sensor system 108, and the display system 110. The control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

The control system 112 may optionally further include a virtual visualization system to provide navigation assistance to operator O when controlling the medical instrument system 104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 may use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

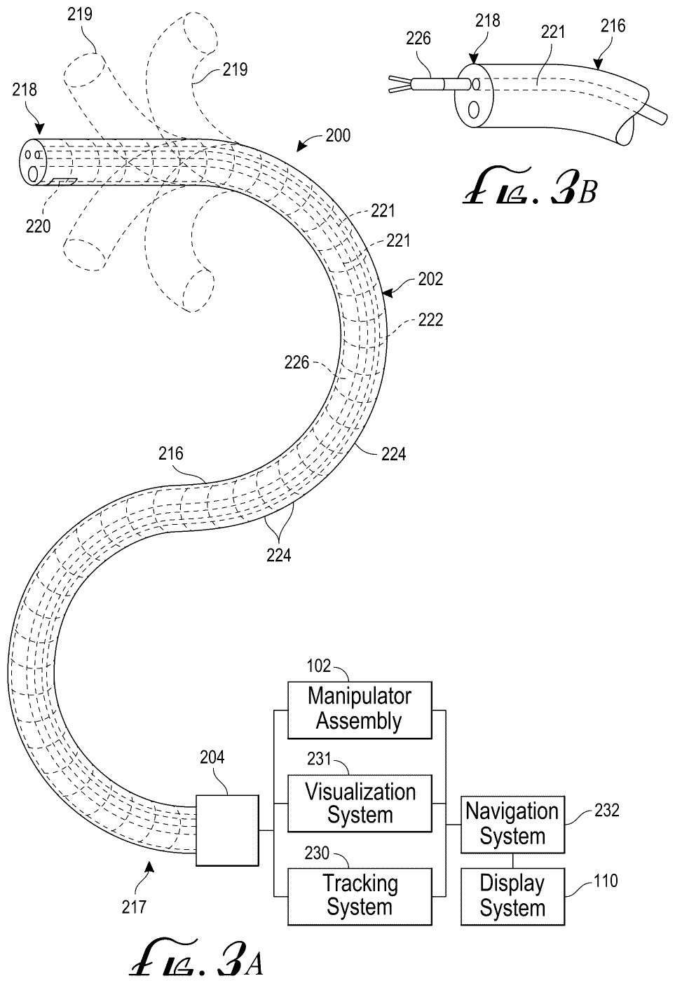

FIG. 3A illustrates a medical instrument system 200 according to some example embodiments. The medical instrument system 200 may be used in an image-guided medical procedure. In some embodiments, the medical instrument system 200 may be used for non-teleoperational exploratory procedures or in procedures involving traditional manually operated medical instruments, such as endoscopy. In other embodiments, the medical instrument system 200 is interchangeable with, or a variation of, the medical instrument system 104 of FIG. 2.

The medical instrument system 200 includes an elongate flexible device 202, such as a flexible catheter, endoscope (e.g., gastroscope, bronchoscope, duodenoscope), or other suitable device coupled to a drive unit 204. The elongate flexible device 202 includes a flexible body 216 having a proximal end portion 217 and a distal end portion 218, including a tip portion. In some embodiments, the flexible body 216 has an outer diameter of approximately 14-20 mm. Other embodiments of the flexible body 216 may have larger or smaller outer diameters. The flexible body 216 may have an appropriate length to reach certain portions of the anatomy, such as the lungs, sinuses, throat, or the upper or lower gastrointestinal region, when the flexible body 216 is inserted into a patient's oral or nasal cavity.

The medical instrument system 200 optionally includes a tracking system 230 for determining the position, orientation, speed, velocity, pose, and/or shape of the distal end portion 218 and/or of one or more segments 224 along the flexible body 216 using one or more sensors and/or imaging devices. The entire length of the flexible body 216, between the distal end portion 218 and the proximal end portion 217, can be effectively divided into segments 224. The tracking system 230 may optionally be implemented as hardware, firmware, software or a combination thereof which interact with or are otherwise executed by one or more computer processors, which may include the processors of the control system 112 in FIG. 2.

The tracking system 230 may optionally track the distal end portion 218 and/or one or more of the segments 224 using a shape sensor 222. In some embodiments, the tracking system 230 may optionally and/or additionally track the distal end portion 218 using a position sensor system 220, such as an electromagnetic (EM) sensor system. In some examples, the position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

The flexible body 216 includes one or more channels (e.g., passageways) sized and shaped to receive one or more medical instruments 226. In some embodiments, the flexible body 216 includes two channels 221 for separate instruments 226, however, a different number of channels 221 may be provided. FIG. 3B is a simplified diagram of an end portion of the flexible body 216 with the medical instrument 226 extended outwardly therefrom according to some embodiments. In some embodiments, the medical instrument 226 may be used for procedures and aspects of procedures, such as surgery, biopsy, ablation, mapping, imaging, illumination, irrigation, or suction. The medical instrument 226 may be deployed through the channel 221 of the flexible body 216 and used at a target location within the anatomy. The medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, expandable ablation members, transducers, sensors, imaging devices and/or the like. The medical instrument 226 may be advanced from the opening of channel 221 to perform the procedure (e.g., electroporation and/or electrolysis in the present disclosure) and then retracted back into the channel 221 when the procedure is complete. The medical instrument 226 may be removed from the proximal end portion 217 of the flexible body 216 or from another optional instrument port (not shown) along the flexible body 216. The medical instrument 226 may be used with an image capture device (e.g., an endoscopic camera) also within the elongate flexible device 202. Alternatively, the medical instrument 226 can itself be the image capture device.

The medical instrument 226 may additionally house cables, linkages, or other actuation controls (not shown) that extend between its proximal and distal end portions 217, 218 to controllably bend the distal end portion 218 of the medical instrument 226. The flexible body 216 may also house cables, linkages, or other steering controls (not shown) that extend between the drive unit 204 and the distal end portion 218 to controllably bend the distal end portion 218 as shown, for example, by the broken dashed line depictions 219 of the distal end portion 218. In some examples, at least four cables are used to provide independent “up-down” steering to control a pitch motion of the distal end portion 218 and “left-right” steering to control a yaw motion of the distal end portion 218. In embodiments in which the medical instrument system 200 is actuated by a robotically-assisted assembly, the drive unit 204 can include drive inputs that removably couple to and receive power from drive elements, such as actuators, of the teleoperational assembly. In some embodiments, the medical instrument system 200 can include gripping features, manual actuators, or other components for manually controlling the motion of the medical instrument system 200. The information from the tracking system 230 can be sent to a navigation system 232 where it is combined with information from the visualization system 231 and/or the preoperatively obtained models to provide the physician or other operator with real-time position information.

In some embodiments, the medical instrument system 200 may be guided manually or via the robotically-assisted manipulator system 100 to deliver the medical instrument 226 to a target tissue site for treatment. In some procedures, the selection between a manual or robotic delivery and the approach may be determined based on the medical application. For example, in some gastrointestinal applications, the medical instrument system 200 (or standalone medical instrument 226) may be delivered via a manual or robotic delivery device endoluminally through a trans-oral or trans-anal approach. A trans-abdominal approach with integrated monopolar or bipolar instrumentation, drop in probes or via catheters may also be used. For urological applications, the medical instrument system 200 (or standalone medical instrument 226) may be delivered via a manual or robotic delivery device endoluminally through a trans-urethral approach, trans-perineal, pre-peritoneal or trans-abdominally with integrated bipolar instrumentation, drop in probes or via catheters. Similarly, for gynecological applications, the medical instrument system 200 (or standalone medical instrument 226) may be delivered via a manual or robotic delivery device endoluminally through a trans-vaginal approach, trans-perineal, or trans-abdominally with integrated bipolar instrumentation, drop in probes or via catheters. For hepatobiliary applications, the medical instrument system 200 (or standalone medical instrument 226) may be delivered via a manual or robotic delivery device endoluminally through a trans-oral approach to reach the ampulla or to go externally into the liver via a trans-gastrointestinal wall route or trans-abdominally with integrated bipolar instrumentation, drop in probes or via catheters. For neurovascular applications, the medical instrument system 200 (or standalone medical instrument 226) may be delivered via a manual or robotic delivery device through an endovascular approach or through a keyhole craniotomy with integrated bipolar instrumentation, drop in probes or via catheters. For cardiac applications, the medical instrument system 200 (or standalone medical instrument 226) may be delivered via a manual or robotic delivery device through an endovascular approach or trans-thoracic with integrated bipolar instrumentation, drop in probes or via catheters.

With collective reference to FIGS. 4A-16D, the following provides additional details of embodiments and features relating to various catheter designs and various components thereof that may be used together with the system 10, the robotically-assisted manipulator system 100, and/or the medical instrument system 200 described previously with reference to FIGS. 1-3B. Generally, the catheters described with reference to FIGS. 4A-16D are designed to facilitate their delivery to the target tissue site and assist with retraction of components after treatment while protecting against potential trauma or damage to surrounding tissue during both catheter delivery and retraction. Other features and advantages of the catheter designs are further described below with reference to the figures. As noted previously, while the following description and related figures relate specifically to catheter designs, other suitable medical instruments (e.g., non-catheter instruments) may be used without departing from the principles of the disclosed subject matter.

FIGS. 4A and 4B collectively illustrate a catheter 300 in a sheathed and unsheathed configuration, respectively, the catheter 300 operable for delivering an electrode array 314 to a target tissue region (e.g., tissue 30 of FIG. 1) in accordance with an example embodiment. The catheter 300 may be used together with the medical instrument systems 104, 200 of FIGS. 2 and 3A, respectively, to provide electrolysis and electroporation treatment according to example embodiments of the present disclosure. With collective reference to FIGS. 4A and 4B, the catheter 300 includes an elongate member 302, such as a shaft or other member have a generally tubular profile, with a distal end 304 and a proximal end 305. The catheter 300 may include a sheath 306 around a portion of the elongate member 302, the sheath 306 having a lumen 308 (see FIG. 4B) extending therethrough. In some embodiments, the length of the elongate member 302 may be at least 85 cm with a preferred length of 180 cm and the diameter of the elongate member 302 may be approximately 5-7 mm with a preferred maximum outer diameter of 6 mm to facilitate introduction of the catheter 300 through the working channel (e.g., working channel 221) of an endoscope or other suitable medical instrument system 200 in a similar fashion as described previously with reference to FIGS. 3A and 3B. It should be understood that in other embodiments, the elongate member 302 may have any suitable dimensions that may depend on the dimensions of the endoscope or other suitable instrument used to introduce the catheter 300 for delivery to target tissue region.

The catheter 300 further includes an expandable member 310 coupled to the elongate member 302. The expandable member 310 may be coupled to the elongate member 302 between the distal end 304 and the proximal end thereof. As illustrated in FIG. 4B, in an unsheathed configuration, the expandable member 310 may be adjacent the distal end 304 in some embodiments. In other embodiments, the expandable member 310 may instead be distal to the elongate member 302 such that no portion of the elongate member 302 extends forward of the expandable member 310.

In the illustrated embodiment, the expandable member 310 is illustrated as an inflatable balloon. In some embodiments, the balloon may be a compliant balloon made of polyurethane, silicone, or other suitable material designed to inflate several times its nominal size. In other embodiments, the balloon may be a semi-compliant balloon made of polyurethane or other suitable material designed for less expansion as compared to a compliant balloon. In other embodiments, other expandable members, such as braided meshes, expandable frames, and the like, may be used.

The expandable member 310 is radially expandable relative to the elongate member 302 and may be expanded in a variety of different manners. For example, in some embodiments, the expandable member 310 may be inflated with a fluid, such as air or another gas, a liquid such as saline, a radiopaque solution, and the like. In some embodiments, the fluid may be introduced to the expandable member 310 through a lumen (not shown) of the elongate member 302. The expandable member 310 may have a length of approximately 4 cm in an unexpanded configuration (as illustrated in FIG. 4B) and may inflate to a diameter between 1 cm and 4 cm. In other embodiments, the length and inflatable diameter of the expandable member 310 may vary depending on the characteristics and dimensions of the target site for tissue treatment. In other examples, the expandable member 310 may be expanded in other suitable ways (e.g., rolling, unfurling, pushing).

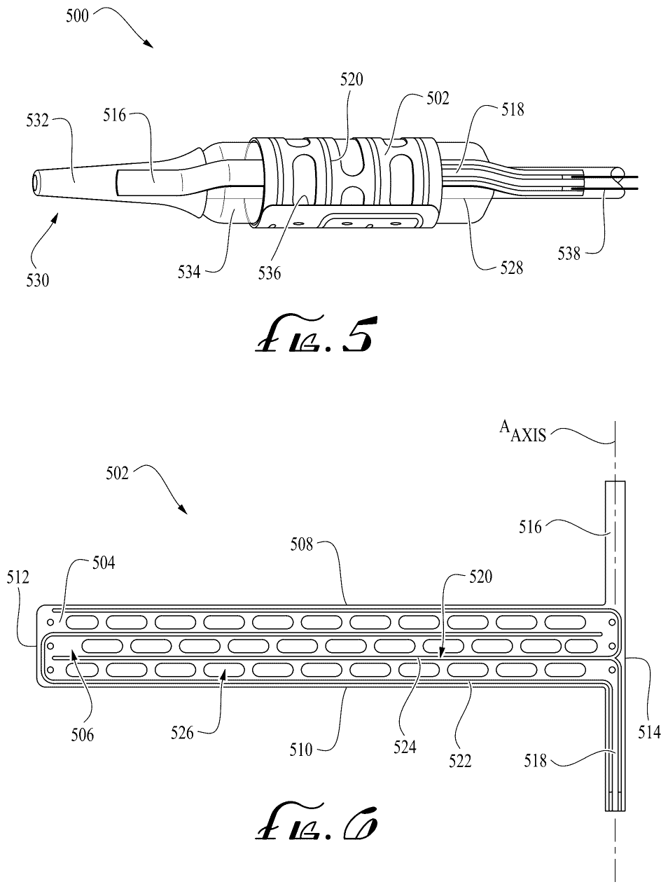

The catheter 300 further includes a flexible substrate 312 that may be coupled to or otherwise supported by the expandable member 310, where the flexible substrate 312 may surround at least a portion of the expandable member 310. In some embodiments, the flexible substrate 312 may be a thin layer or sheet comprised of an electrically insulating polymer material, such as polyimide, polyester, or other suitable thermoplastic or thermosetting polymer film. In other embodiments, the flexible substrate 312 may comprise a polymer covered material. Additional details of various components, features, and embodiments of the flexible substrate 312 are provided below with particular reference to FIGS. 5-6.

The catheter 300 includes an electrode array 314 deposited on or otherwise supported by the flexible substrate 312, where the electrode array 314 is suitable for providing one or both of electrolysis and electroporation to the targeted tissue in a similar fashion as described previously. Accordingly, the electrode array 314 may take any suitable configuration operable for generating an electric field. For example, in one embodiment, the electrode array 314 may comprise a plurality of metal traces deposited on the flexible substrate 312. The metal traces may include any suitable conductive material such as gold, copper, stainless steel, titanium, graphene, graphite, and the like. Generally, electrode materials may be selected such that the electrode material does not actively participate in electrolysis products and/or leave material residue. In some embodiments, the electrode material may be chosen to minimize transferring ions from the electrode material to the target tissue. For example, steel may be less preferred. Titanium and gold may have some degree of participation in the electrolysis process, however they may not generate a toxic residue and may be used in some examples.

Generally, the electrodes used in the example configurations described herein, particularly those described with reference to FIGS. 5-16D, are arranged in a bipolar configuration. Generally, in a bipolar configuration, current may travel from one electrode of a pair of electrodes to another electrode of the pair of electrodes. The electrodes in the pair may accordingly be said to be of opposite polarity. In some configurations, multiple pairs of electrodes may be used, with the current passing through each pair of electrodes. Any shape of electrodes may be used in the example configurations described herein including circular, square, rectangular, or other shapes. Interdigitated electrodes may also be used.

In other examples, the electrodes may be arranged in a monopolar configuration or may be arranged in a combination of bipolar and monopolar configurations. A monopolar configuration may include an active electrode (e.g., an electrode on, in, or near the target tissue) and a return electrode. The return electrode may be placed away from the target tissue but in contact with the patient in some examples (e.g., using a pad having an electrode). In this manner, one polarity (e.g., the polarity of the active electrode) is in contact with or near the target tissue. Accordingly, a monopolar configuration of electrodes may include a pair of electrodes—with one energized and one serving as a return. The return electrode may not be provided on the same device or a separate device as the energized electrode. For example, the return electrode may be provided on a pad placed proximate the tissue. In a monopolar configuration, multiple active electrodes may be present and may pass current through a shared return electrode in some examples, or through respective return electrodes in other examples.

As illustrated in FIG. 4B, the catheter 300 includes wiring 316 that runs along the elongate member 302 to provide electrical signals, such as voltages and/or currents, to the electrode array 314. In one embodiment, the wiring 316 may run down the length of the elongate member 302 to the controller 14 and/or power supply 12 (see FIG. 1), which may be outside the body. In other embodiments, other suitable wiring configurations may be used to power the electrode array 314.

With general reference to FIGS. 1-4B, the following provides a brief overview of an example delivery of the catheter 300 to the duodenum for ablation of mucosal lining in accordance with an example embodiment. In one example, the medical instrument system 200 (e.g., endoscope) may be delivered in a trans-oral manner to the duodenum, which includes entry through the patient's esophagus, navigation through the stomach, and finally into the duodenum for treatment. With the medical instrument system 200 in position, the catheter 300 is navigated through the working channel 221 and into position within the duodenum. Thereafter, the expandable member 310 is inflated to radially expand the expandable member 310 to a target diameter. Expansion of the expandable member 310 exerts radial forces against the flexible substrate 312, which urge the flexible substrate 312 to unfurl and/or expand toward the mucosal lining of the duodenum. As the flexible substrate 312 deploys, the electrode array 314 on the flexible substrate 312 is moved into position adjacent to (or in contact with) the mucosal lining. Expansion of the expandable member 310 and/or the flexible substrate 312 may be controlled to ensure that the electrode array 314 appropriately contacts (or is otherwise positioned with sufficient proximity) for treatment of the mucosal lining while avoiding overexpansion. With the electrode array 314 in proper position, a current is run through the wiring 316 and to electrode array 314 to generate an electric field for electroporation and/or electrolysis. Preferably, the area to be ablated is localized to the region around the expandable member 310, and the electric field is targeted to ablate the mucosal lining of the duodenum without affecting adjacent tissues. Upon completion of treatment, the expandable member 310 is deflated to retract the flexible substrate 312 and electrode array 314 and move them away from the tissue and back into the stowed position to facilitate removal of the catheter 300.

In some embodiments, the catheter 300 and other the catheters described herein may be actuated by a robotically-assisted manipulator system (e.g., manipulator assembly 102). The catheter may include or be coupled to a drive unit (e.g., drive unit 204) having one or more drive inputs that removably couple to and receive power from drive elements, such as actuators, of the manipulator assembly. The drive inputs of the catheter may be coupled to drive outputs of the manipulator assembly. In addition, the drive inputs of the catheter may be coupled to the distal end portion of the catheter via one or more actuation drive members (e.g., actuation cables, actuation rods, tension members, and the like) to perform actions such as advancing, retracting, or articulating the distal end of the catheter as well as expansion and/or retraction of the expandable members described herein to adjust the flexible substrate between the deployed position and the stowed position. In some embodiments, the shaft of the elongate member may be coupled to the drive unit for advancing and retracting the shaft. In addition, the drive unit may be coupled to a controller (e.g., controller 14) and/or power supply (e.g., power supply 12) to provide electrical power to electrodes on the flexible substrate. The drive unit may be electrically coupled to the flexible substrate via one or more electrical cables running along or through the shaft of the catheter. In alternative embodiments, the controller and power supply may provide power to the electrical cables without coupling to the drive unit (e.g., the controller and power supply may be independent of the drive unit).

As noted previously, in some embodiments, the catheter 300 may be repositioned (e.g., advanced or retracted relative to the medical instrument system 200) to a different region of the duodenum and the process repeated as needed to sequentially treat a larger region of the mucosal lining. It should be understood that while the example above illustrates the use of a delivery system including an endoscope to deliver a catheter to the treatment area, other delivery systems may additionally or instead be used in other embodiments. In still other embodiments, the catheter 300 may be delivered on its own without aid of an endoscope or other delivery system. Further, it should be understood that treatment in the duodenum is one example for delivery of the catheter 300 and that the catheter 300 may be used for other target tissues.

FIG. 5 illustrates another catheter 500 with a flexible substrate 502 and an electrode array 520 for delivery to a target tissue region in accordance with an example embodiment. FIG. 6 illustrates the flexible substrate 502 of the catheter 500 in accordance with an example embodiment. Generally, the catheter 500 is arranged in a similar fashion as the catheter 300 of FIGS. 4A and 4B. Accordingly, the following description of catheter 500 may not provide additional detail for various like components to avoid repetition with the understanding that those components may have the same or substantially similar features as the corresponding components of catheter 300.

With particular reference to FIG. 6, the flexible substrate 502 includes a base 504 with a top surface 506 and an opposite bottom surface (not shown). The base 504 may be a thin layer or sheet comprised of an electrically insulating polymer material, such as polyimide, polyester, or other suitable thermoplastic or thermosetting polymer film, or may comprise a base material with one or more coating layers comprising a suitable electrically insulating polymer. The base 504 includes a header or distal portion 508, an opposite footer or proximal portion 510, a first peripheral side portion 512, and a second peripheral side portion 514. The peripheral side portions 512, 514 each span between the header portion 508 and the footer portion 510 along opposite ends of the base 504.

The base 504 of the flexible substrate 502 further includes a first arm segment 516 extending outwardly from the header portion 508 and a second arm segment 518 extending outwardly from the footer portion 510. The arm segments 516, 518 may each extend substantially orthogonal to the respective header portion 508 and to the footer portion 510. In some embodiments, the arm segments 516, 518 are aligned relative to one another along a first axis A extending across the flexible substrate 502 and traversing both the header portion 508 and the footer portion 510. For example, the arm segments 516, 518 may be aligned relative to one another along the second peripheral side portion 514 of the flexible substrate 502. This configuration provides the flexible substrate 502 with a generally T-shaped configuration as illustrated in FIG. 6. Alternatively, the arm segments 516, 518 may instead be aligned along the first peripheral side portion 512. In other embodiments, one of the arm segments 516, 518 may extend from the header portion 508 and the other of the arm segments 516, 518 may extend from the footer portion 510 but not in alignment relative to one another. In other words, the arm segments 516, 518 are not aligned along a common axis extending across the flexible substrate 502 from the header and footer portions 508, 510. In some embodiments, the arm segments 516, 518 are each formed as integral portions of the flexible substrate 502, but one or both could also be formed as separate components from the flexible substrate 502 that may be coupled (e.g., via adhesive or other suitable bonding mechanisms) in a similar configuration described above.

The flexible substrate 502 includes an electrode array 520 disposed along the top surface 506 of the base 504. As described previously, the electrode array 520 may take any suitable configuration operable for generating an electric field to provide electroporation and/or electrolysis for tissue treatment. For example, in the embodiment of FIG. 6, the electrode array 520 comprise a plurality of metal traces 522, 524 deposited on the flexible substrate 502, where the metal traces 522, 524 include any suitable electrically conductive material, such as gold, copper, stainless steel, titanium, graphene, graphite, and the like. In one embodiment, at least a portion of the electrode array 520 extends onto one of the arm segments (arm segment 518 in FIG. 6), with the other of the arm segments (arm segment 516 in FIG. 6) being substantially free of the electrode array 520. In other embodiments, the electrode array 520 may extend onto both of the arm segments 516, 518 or onto neither of the arm segments 516, 518.

The base 504 of the flexible substrate 502 may further optionally include a plurality of apertures 526 formed thereon to provide additional flexibility for the flexible substrate 502 (and the overall design of the catheter 500) to facilitate delivery through an endoscopic working channel in a similar fashion as described with reference to FIGS. 2-3B. In some embodiments, at least some of the apertures 526 are disposed between portions of the electrode array 520 to avoid or minimize impact on the ability to generate a desired electric field for treatment purposes. For example, with reference to FIG. 6, the electrode array 520 may include a first trace element 522 and a second trace element 524 offset from one another along the top surface 506 of the base 504. In such embodiments, at least some of the apertures 526 are disposed between the trace elements 522, 524 on the flexible substrate 502. In other embodiments, the apertures 526 may be arranged differently to provide desired flexibility to the flexible substrate 502 for navigating tortuous structures within the body. In other embodiments, the apertures 526 may be omitted.

The flexible substrate 502 may include any suitable dimensions to achieve the purposes described herein. For example, in one embodiment, the base 504 may have a length from the first peripheral side portion 512 to the second peripheral side portion 514 ranging between 60-180 mm. The base 504 may have a width from the header portion 508 to the footer portion 510 (excluding the arm segments 516, 518) ranging between 7-60 mm. The arm segments 516, 518 may each range from 2-45 mm (measured from the respective header and footer portions 508, 510). The base 504 may have a thickness ranging between 12-254 μm, and the electrode array 520 may have a thickness ranging from 12-102 μm.

With reference to FIGS. 5 and 6, the following describes an example coupling arrangement of the flexible substrate 502 with an expandable member 534 in accordance with one embodiment. Briefly, with reference to FIG. 5, the catheter 500 includes an elongate member 528 with a distal portion 530 and a proximal portion (not shown). In some embodiments, the distal portion 530 may include a distal tip 532 designed to help guide the catheter 500 to the target tissue region. The catheter 500 further includes an expandable member 534 (e.g., an inflatable balloon, mesh, or other suitable expandable member) coupled to the elongate member 528. The expandable member 534 may be coupled to the elongate member 528 between the distal portion 530 and the proximal portion thereof, where the expandable member 534 is radially expandable relative to the elongate member 528 as described previously with reference to catheter 300.

The flexible substrate 502 is coupled to the expandable member 534 and is expandable or otherwise deployable therewith in a similar fashion as described previously. With reference to FIG. 5, the flexible substrate 502 tightly surrounds at least a portion of the expandable member 534 when the flexible substrate 502 is in a stowed position. In some embodiments, the flexible substrate 502 may be wrapped around the expandable member 534, with segments of the flexible substrate 502 overlapping onto other segments of the flexible substrate 502. When in the stowed position against the expandable member 534, the flexible substrate 502 may have an outer diameter ranging from 5-20 mm. The outer diameter is preferably kept as small as possible when in the stowed position to facilitate delivery and retraction of the catheter 500 during and after treatment. For example, the outer diameter in the stowed configuration may be sized to fit within the working channel 221 of the medical instrument system 200 (e.g., an endoscope). Once expanded, the flexible substrate 502 may have an outer diameter ranging from 6-50 mm in some embodiments, and from 20-50 mm in some embodiments. In some embodiments, the outer diameter may be based on the tissue profile being treated and/or the expansion specifications of the expandable member 534.

In some embodiments, the flexible substrate 502 may be processed to enable appropriate expansion of the flexible substrate 502 under pressure (e.g., when the expandable member 534 is expanded), and to ensure that the flexible substrate 502 returns to its collapsed state (e.g., the stowed configuration) after the pressure source is removed (e.g., when the expandable member 534 is deflated). For example, in one embodiment, the flexible substrate 502 may undergo a shape-setting process into a rolled configuration using heat and pressure by approaching the glass-transition temperature of the material comprising the flexible substrate 502. The flexible substrate 502 may also be cooled under pressure to hold shape memory in a rolled configuration. Initially shaping the flexible substrate 502 into a rolled configuration urges the flexible substrate 502 to return to the rolled configuration after deployment to facilitate retraction.

In some embodiments, certain aspects of the flexible substrate 502 may also be treated to encourage specific characteristics as desired. For example, the flexible substrate 502 may have a tail portion 536 (see FIG. 5) formed along the outer end of the flexible substrate 502 (e.g., along peripheral side portion 512) after the flexible substrate 502 is wrapped around the expandable member 534 as described previously. Depending on the material composition and/or other characteristics of the flexible substrate 502, the tail portion 536 may tend to curl inwardly against the expandable member 534 and/or may urge the flexible substrate 502 to partially or completely unfurl when in the stowed configuration. Accordingly, in one embodiment, the tail portion 536 may be heated to straighten the tail portion 536 out after the flexible substrate 502 is shaped in a rolled configuration as described previously. In some embodiments, the straightened tail portion 536 may also help resist the flexible substrate 502 from rolling into multiple barrels (e.g., one barrel around the expandable member 534 and a second barrel adjacent the expandable member 534). The flexible substrate 502 may tend to roll into multiple barrels if over-expanded during the deployment phase.

In other embodiments, other shape-setting techniques may be applied to the flexible substrate 502 to ensure it retains its shape around the expandable member 534 during delivery in the stowed configuration, during deployment in the expanded configuration, and during retraction back to the stowed configuration for removal after treatment.

With the flexible substrate 502 in the wrapped and stowed configuration, one arm segment (first arm segment 516 in FIG. 5) extends outwardly from the flexible substrate 502 and toward the distal portion 530 of the elongate member 528. The first arm segment 516 is coupled to the expandable member 534 to retain the flexible substrate 502 in position against the expandable member 534 in the stowed position. In some embodiments, the first arm segment 516 may also (or alternatively) be coupled to the distal tip 532. The first arm segment 516 helps resist the flexible substrate 502 from peeling off or away of the expandable member 534 in the proximal direction when the flexible substrate 502 encounters resistance during catheter delivery, positioning, or retraction. The first arm segment 516 may be coupled to the expandable member 534 (and/or distal tip 532) using any suitable technique, such as bonding agents or suitable adhesives. In some embodiments, the first arm segment 516 remains coupled to the expandable member 534 after the expandable member 534 is inflated to deploy the flexible substrate 502. In other embodiments, the first arm segment 516 may dislodge from the expandable member 534 during deployment of the flexible substrate 502. As illustrated in FIG. 5, the catheter 500 includes one or more electrical wires 538 coupled to the electrode array 520 along a proximal portion of the flexible substrate 502, where the wires 538 run toward the proximal portion of the elongate member 528 and are coupled to a power supply (e.g., power supply 12 of FIG. 1). The wires 538 may be coupled to the flexible substrate 502 along the second arm segment 518 to help retain the flexible substrate 502 in position against the expandable member 534 along a proximal portion of the elongate member 528 during catheter delivery and retraction.

In some embodiments, as described previously, the first arm segment 516 of the flexible substrate 502 coupled along the distal portion 530 is substantially free of any electrodes of the electrode array 520 since it primarily serves as a supporting tie down to retain the flexible substrate 502 in position, whereas the second arm segment 518 may include portions of the electrode array 520 for coupling the wires 538 to generate the electric field for treatment. It should be understood that while the embodiment illustrates and describes the first arm segment 516 coupled along the distal portion 530 of the elongate member 528 and the second arm segment 518 coupled along the proximal portion of the elongate member 528, the positioning of the arm segments 516, 518 may be swapped in other embodiments.

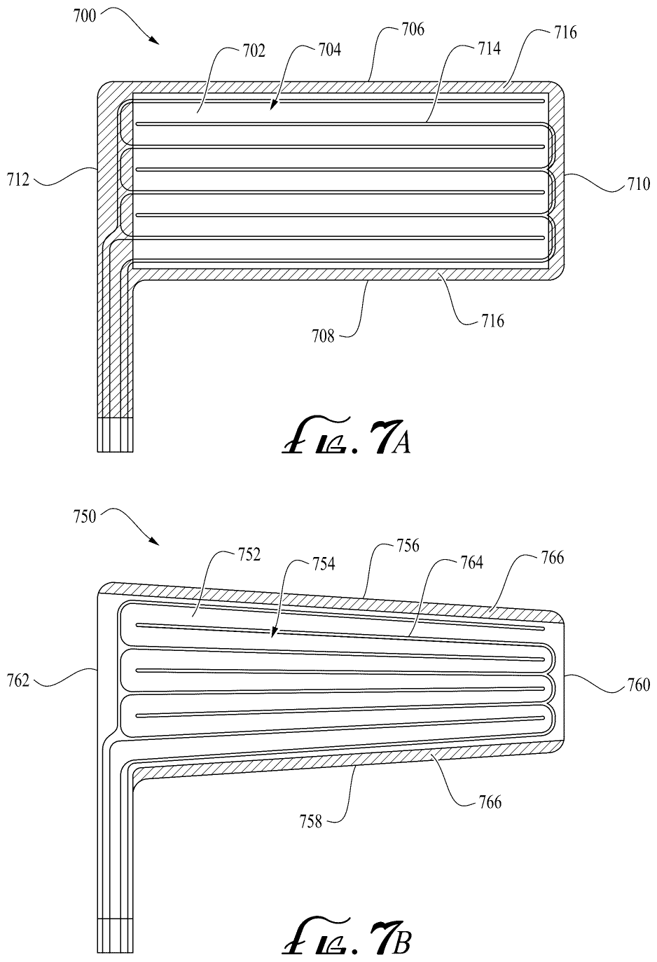

In some embodiments, the header portion 508 and the footer portion 510 of the flexible substrate 502 may form relatively hardened, sharp, and/or stiff leading and trailing edges that may rub against or otherwise disturb tissue during delivery and/or retraction of the catheter 500 when the flexible substrate 502 is in the stowed configuration. Accordingly, the edges may inadvertently damage tissue around the treatment site. With collective reference to FIGS. 7A-10, the following disclosure provides various potential solutions for minimizing trauma during catheter delivery and retraction.

FIG. 7A illustrates another embodiment of a flexible substrate 700 that may be used with the catheters 300, 500 described above in accordance with an example embodiment. Generally, the flexible substrate 700 includes a base 702 with a top surface 704 and an opposite bottom surface (not shown). The base 702 may be comprised of an electrically insulating polymer material, such as polyimide, polyester, or other suitable thermoplastic or thermosetting polymer film in a similar fashion as the base 504 of the flexible substrate 502 of FIG. 6. The base 702 includes a header portion (e.g., distal portion) 706, an opposite footer portion (e.g., proximal portion) 708, a first peripheral side portion 710, and a second peripheral side portion 712. The peripheral side portions 710, 712 each span between the header portion 706 and the footer portion 708 along opposite ends of the base 702. The base 702 further includes an electrode array 714 on the top surface 704.

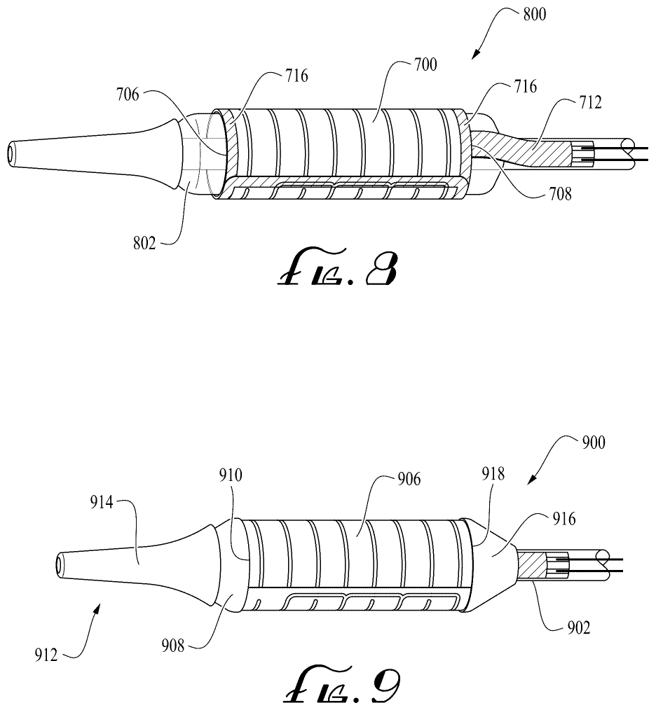

In one embodiment, the base 702 of the flexible substrate 700 may include a protective coating layer 716 covering the outer edge surface of the header portion 706 and the footer portion 708 and/or of the respective peripheral side portions 710, 712. In some embodiments, the protective coating layer 716 may extend onto a portion of the top surface 704 and the opposite bottom surface and may also extend beyond the respective outer edge surfaces of the base 702 of the flexible substrate 700. The protective coating layer 716 may be any suitable material, such as silicone or other soft polymer, and may have any suitable thickness as desired to provide an atraumatic surface to the peripheral side portions 710, 712 (and to the header and footer portions 706, 708) of the flexible substrate 700 as further described in detail below with reference to FIG. 8. For example, in one embodiment, the protective coating layer 716 may be 1 mm in thickness or may range in thickness between 0.25 mm to 2 mm or may have other suitable dimensions. The protective coating layer 716 may be applied or deposited onto the flexible substrate 700 in any suitable manner. For example, in one embodiment, the protective coating 716 may be overmolded onto the flexible substrate 700 as part of a post-processing procedure. In some embodiments, a nano spray coating (or other suitable coating) may be applied to reduce friction of the flexible substrate 700.

In some embodiments, the protective coating layer 716 (or a different protective coating layer not shown) may cover some or all of the top surface 704 and/or bottom surface of the flexible substrate 700 to further protect against potential tissue damage from any aspect of the flexible substrate 700. The protective coating layer 716 may also cover some or all of the electrode array 714 disposed on the top surface 704 of the flexible substrate 700. In these embodiments, the protective coating layer 716 may include silicone, parylene, or other suitable coatings. Both silicone and parylene may act as suitable insulators in cases where the electrode array 714 is under-deployed to minimize potential complications. In some embodiments, the inner lubricity created by the protective coating layer 716 may facilitate smooth deployment and unfurling of the flexible substrate 700, as it may act to reduce friction between overlapping sections of the flexible substrate 700, especially where the material comprising the flexible substrate 700 may be somewhat sticky or tacky. In addition, the protective coating layer 716 may also help prevent the electrode array 714 from sticking against the tissue and/or itself when being retracted from the deployed configuration back to the furled, stowed configuration for removal. In some embodiments, additional sprays or other substances may be applied to the flexible substrate 700 to reduce friction as needed to ensure the flexible substrate 700 deploys as desired.

FIG. 8 illustrates an example catheter 800 using with the flexible substrate 700 of FIG. 7A in accordance with one embodiment. The catheter 800 may have the same or substantially similar features with similar functionality as described previously with respect to the catheters 300, 500. Accordingly, additional details of the catheter 800 may not be further described in detail below to avoid repetition and/or obscuring more pertinent details of the embodiment. With particular reference to FIG. 8, when the flexible substrate 700 is coupled to the expandable member 802, the header and footer portions 706, 708 form leading (e.g., distal) and trailing (e.g., proximal) edges, respectively, of the flexible substrate 700 that may be relatively hardened, sharp, and/or stiff. In this configuration, the flexible substrate 700 may cause inadvertent damage to tissue during catheter delivery and treatment as noted previously. By coating the header and footer portions 706, 708 (or at least coating the header portion 706 that forms the leading edge of the flexible substrate 700 during catheter delivery) with a protective coating layer 716 as described, the risk for traumatic injury is mitigated since the leading and/or trailing edges are coating with a soft material relative to the material comprising the flexible substrate 700.

As described above with reference to FIGS. 7A and 8, in some embodiments, some or all of the outer edge surfaces of the flexible substrate 700 may be covered with a protective coating layer 716 to reduce potential trauma to surrounding tissues when the flexible substrate 700 is in a stowed configuration during catheter delivery. However, in some embodiments, the protective coating layer 716 around the edges of the flexible substrate 700 increases the overall thickness of the flexible substrate 700 when in the stowed configuration since the protective coating layer 716 stacks upon itself as the flexible substrate 700 is wrapped around the expandable member 802. This stacking effect may also impact how tightly the flexible substrate 700 may be wrapped against the expandable member 802 when in the stowed configuration. In some embodiments, the thickness of the protective coating layer 716 may create raised portions of the flexible substrate 700 and may reduce the opening force of the flexible substrate 700 due to capstan effect. Accordingly, in some embodiments, the design of the header and footer portions of the flexible substrate 700 may be altered to minimize stacking of the protective coating layer 716 as described below with reference to FIG. 7B.

FIG. 7B illustrates another embodiment of a flexible substrate 750 that may be used with any of the catheters 300, 500, 800 described above (or with other suitable catheter designs) in accordance with an example embodiment. Generally, the flexible substrate 750 includes many of the same features as the flexible substrate 700 described with reference to FIG. 7A. For example, with reference to FIG. 7B, the flexible substrate 750 includes a base 752 with a top surface 754 and an opposite bottom surface (not shown). The base 752 may be comprised of an electrically insulating polymer material, such as polyimide, polyester, or other suitable thermoplastic or thermosetting polymer film in a similar fashion as the base 702 of the flexible substrate 700 of FIG. 7A. The base 752 includes a header portion (e.g., distal portion) 756, an opposite footer portion (e.g., proximal portion) 758, a first peripheral side portion 760, and a second peripheral side portion 762. The peripheral side portions 760, 762 each span between the header portion 756 and the footer portion 758 along opposite ends of the base 752. The base 752 further includes an electrode array 764 on the top surface 754.

In one embodiment, the header portion 756 and the footer portion 758 may be tapered, where the header portion 756 and the footer portion 758 are angled relative to one another. For example, in one embodiment, the width of the base 752 of the flexible substrate 750 may gradually narrow along the header portion 756 and the footer portion 758 from the second peripheral side portion 762 toward the first peripheral side portion 760. In this configuration, a length of the second peripheral side portion 762 is greater than a respective length of the first peripheral side portion 760 as illustrated in FIG. 7B. In this tapered configuration, when the flexible substrate 750 is wrapped around an expandable member (e.g., expandable member 802 of FIG. 8) into a stowed configuration, the tapered header and footer portions 756, 758 result in the protective coating layer 766 not stacking upon itself as the flexible substrate 750 is tightly rolled. Instead, layers of the protective coating layer 766 along the header and footer portions 756, 758 sit inside of and do not overlap successive layers as the flexible substrate 750 is wrapped.

FIG. 9 illustrates another embodiment of a catheter 900 with features designed for preventing trauma during catheter delivery and retraction. Generally, the catheter 900 may have the same or substantially similar features as the catheters 300, 500. Accordingly, certain details of the catheter 900 may not be further described in detail below to avoid repetition and/or obscuring more pertinent details of the embodiment. Briefly, with reference to FIG. 9, the catheter 900 includes an elongate member 902, an expandable member (obscured from view in FIG. 9) coupled thereto, and a flexible substrate 906 coupled to the expandable member in a similar fashion and with similar functionality as described previously with respect to catheters 300, 500.

With reference to FIG. 9, the catheter 900 may include a first cushion 908 disposed adjacent a leading edge 910 (which may be defined along one of the header portion or the footer portion of the flexible substrate 906 in a similar fashion as described previously with reference to FIG. 8) of the flexible substrate 906, where the first cushion 908 is primarily designed to protect against tissue trauma during delivery of the catheter 900. The first cushion 908 may contact the leading edge 910 of the flexible substrate 906 to ensure there is no gap therebetween and avoid potentially catching or trapping tissue therein. In some embodiments, the first cushion 908 may be positioned adjacent a distal portion 912 or between the distal portion 912 and the flexible substrate 906 of the catheter 900. The first cushion 908 may be made of any suitable material, such as a cling-type film material or other soft material, for providing a soft interface and obscuring the leading edge 910 of the flexible substrate 906 to minimize contact or interaction of the leading edge 910 with tissue primarily during catheter delivery (but also protects tissue during retraction).

In some embodiments, the first cushion 908 may be coupled to the elongate member 902, to a distal tip 914 of the elongate member 902, to the expandable member, and/or to the flexible substrate 906. In some embodiments, the first cushion 908 is coupled in such a manner so that it is retained in position against the catheter 900 during and after deployment of the flexible substrate 906 for treatment. Accordingly, the first cushion 908 may be made of such material with sufficient flexibility so as to not impair either delivery of the catheter 900 or deployment and retraction of the expandable member and the flexible substrate 906. In other embodiments, the first cushion 908 may be a silicone, polyurethane, or other suitable polymer coating that is weakly attached to the flexible substrate 906 and designed to peel off during expansion of the expandable member and the flexible substrate 906. In still other embodiments, the first cushion 908 may be made as a standalone cap designed for subsequent attachment to the catheter 900. In some embodiments, the first cushion 908 may be a coating designed to keep the leading edge 910 low profile until expansion of the flexible substrate 906, where the coating is designed to act like a weak, temporary adhesive that rips or peels easily during expansion.

In some embodiments, the catheter 900 includes a second cushion 916 disposed adjacent a trailing edge 918 (which may be defined along the other of the header portion or the footer portion of the flexible substrate as described previously) of the flexible substrate 906. The second cushion 916 may contact the trailing edge 918 of the flexible substrate 906 to ensure there is no gap therebetween. The second cushion 916 may be made of any suitable material, such as a cling-type film material or other soft material, for providing a soft interface and obscuring the trailing edge 918 of the flexible substrate 906 to minimize contact or interaction of the trailing edge 910 with tissue primarily during catheter retraction (but also protects tissue during delivery). The second cushion 916 may otherwise be substantially similar to the first cushion 908 described above. Accordingly, the features and characteristics of the first cushion 908 described above may apply equally to the second cushion 916 but are not further discussed in detail to avoid repetition.

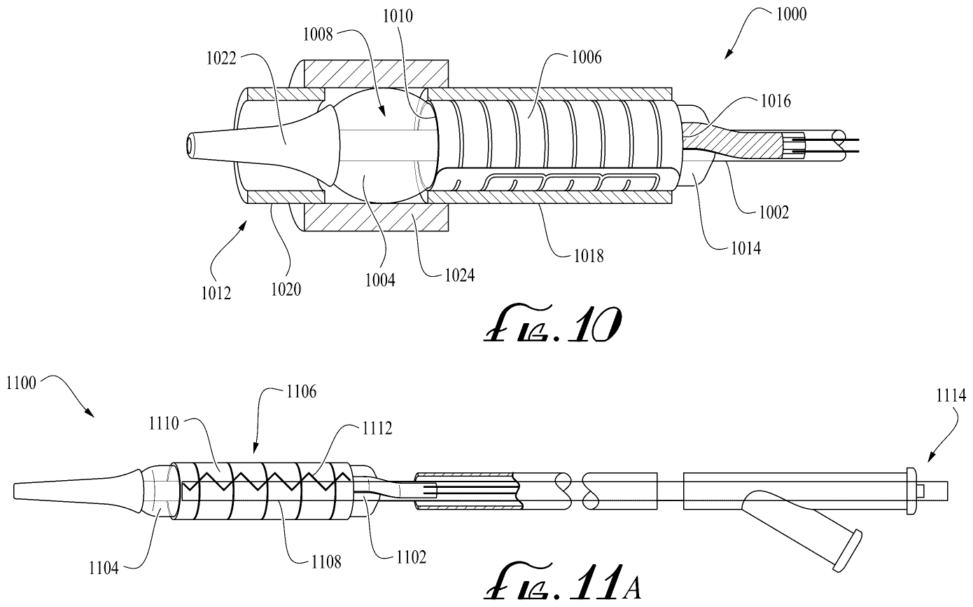

FIG. 10 illustrates another embodiment of a catheter 1000 designed with features designed for preventing trauma during catheter delivery, positioning, and retraction. Generally, the catheter 1000 may have the same or substantially similar features as the catheters 300, 500. Accordingly, certain details of the catheter 1000 may not be further described in detail below to avoid repetition and/or obscuring more pertinent details of the embodiment. Briefly, with reference to FIG. 10, the catheter 1000 includes an elongate member 1002, an expandable member 1004 coupled thereto, and a flexible substrate 1006 coupled to the expandable member 1004 in a similar fashion and with similar functionality as described previously with respect to catheters 300, 500.

With reference to FIG. 10, the catheter 1000 may include a first cushion 1008 disposed adjacent a leading edge 1010 (which may be defined along one of the header portion or the footer portion of the flexible substrate as described previously) of the flexible substrate 1006, where the first cushion 1008 is primarily designed to protect against tissue trauma during delivery of the catheter 1000. The first cushion 1008 may contact the leading edge 1010 of the flexible substrate 1006 to ensure there is no gap therebetween. The first cushion 1008 may be positioned adjacent a distal portion 1012 or between the distal portion 1012 and the flexible substrate 1006 of the catheter 1000. As illustrated in FIG. 10, the first cushion 1008 may be formed by partially expanding or inflating the expandable member 1004 such that the first cushion 1008 sufficiently covers or obscures the leading edge 1010 of the flexible substrate 1006 to minimize contact or interaction of the leading edge 1010 with tissue primarily during catheter delivery (but also protects tissue during retraction).

In some embodiments, the catheter 1000 may also include a second cushion 1014 (shown in an unexpanded configuration) disposed adjacent a trailing edge 1016 (which may be defined along the other of the header portion or the footer portion of the flexible substrate as described previously) of the flexible substrate 1006. The second cushion 1014 may contact the trailing edge 1016 of the flexible substrate 1006 to ensure there is no gap therebetween. In a similar fashion as the first cushion 1008, the second cushion 1014 may be formed by partially expanding or inflating the expandable member 1004 such that the second cushion 1014 sufficiently covers or obscures the trailing edge 1016 of the flexible substrate 1006 to minimize contact or interaction of the trailing edge 1016 with tissue primarily during catheter retraction (but also protects tissue during delivery).

The cushions 1008, 1014 may be made in any suitable fashion. For example, in one embodiment, a first tube 1018 may be positioned over the flexible substrate 1006 and a second tube 1020 may be positioned over a distal tip 1022 of the elongate member 1002. The first and second tubes 1018, 1020 are offset from one another, leaving a small gap separating the tubes 1018, 1020. Another tube 1024 of a larger diameter than the tubes 1018, 1020 may be positioned over the gap and the tubes 1018, 1020 to enclose a shoulder around an exposed portion of the expandable member 1004. The tubes 1018, 1020, 1024 may be of any suitable material, such as tetrafluoroethylene (TFE) and the like. With the tubes 1018, 1020, 1024 in position as illustrated in FIG. 10 for example, the exposed portion of the expandable member 1004 may be heated under pressure to urge it to fill the gap and create the first cushion 1008. A similar process may be used to create the second cushion 1014 along the trailing edge 1016. Once the cushions 1008, 1014 have been created, the tubes 1018, 1020, 1024 may be removed to maintain a low overall profile for the catheter 1000 and facilitate catheter delivery to and from the target tissue site. In another embodiment, the expandable member 1004 may be pre-set and/or pre-expanded to form the cushions 1008, 1014. In such embodiments, the expandable member 1004 may include a thermoset material to facilitate forming the cushions 1008, 1014 during a manufacturing process.

In some embodiments, the expandable member 1004 may return to its nominal shape (e.g., the cushions 1008, 1014 may be dislodged or removed) after the expandable member 1004 is inflated for deploying the flexible substrate 1006. In other embodiments, the cushions 1008, 1014 may be permanently retained such that they are in position against the leading and trailing edges 1010, 1016, respectively, after the expandable member 1004 is deflated and the flexible substrate 1006 is retracted to the stowed position to protect the tissue during catheter removal.

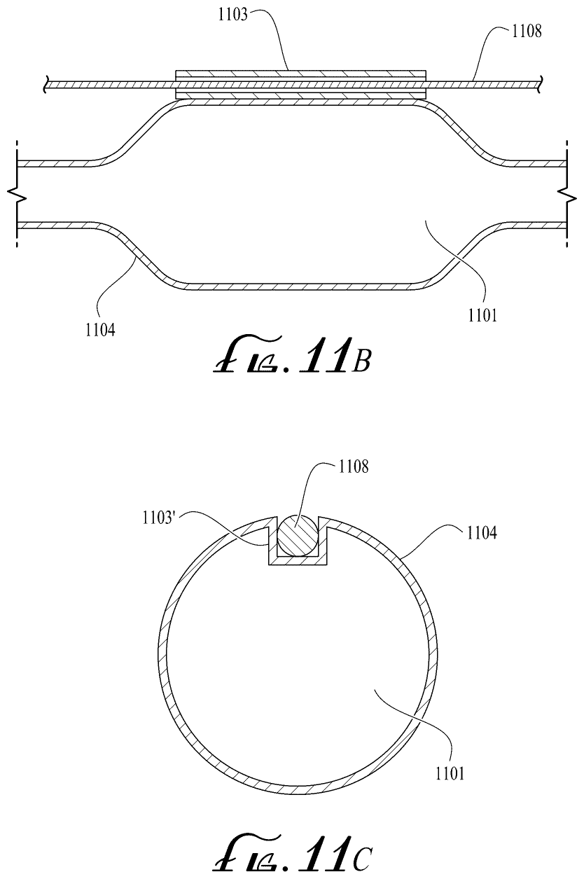

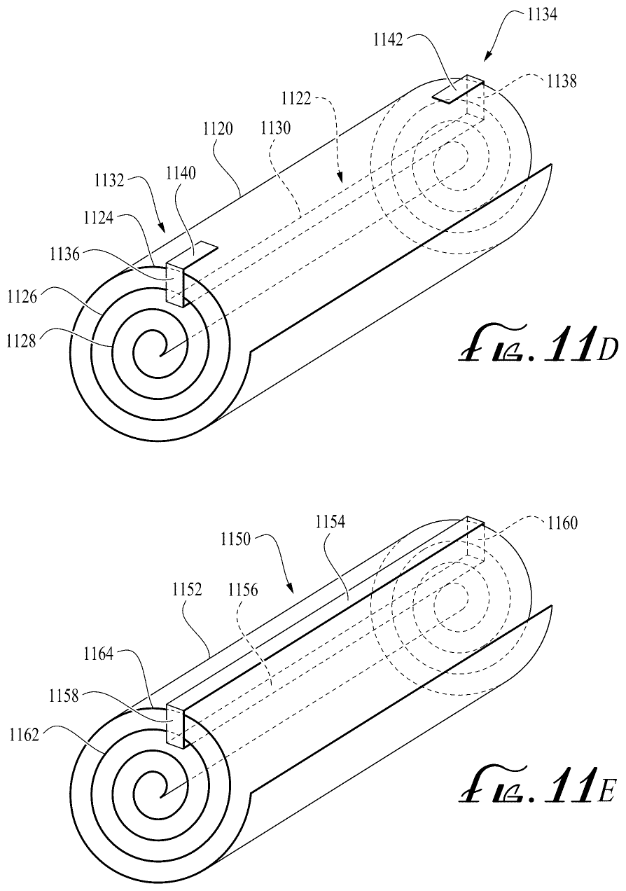

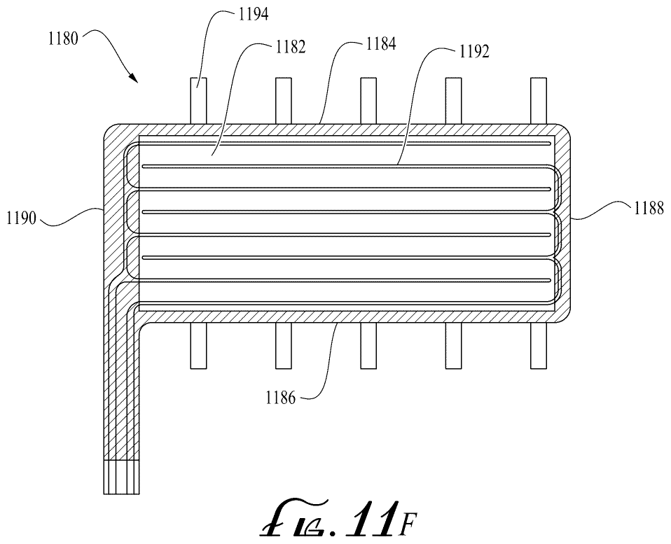

FIG. 11A illustrates a catheter 1100 with an adjustment member 1108 operable to retain a flexible substrate 1106 in a stowed position during delivery to a target region in accordance with examples described herein. The adjustment member 1108 may additionally be operable to transition the flexible substrate 1106 from an expanded configuration (e.g., from a deployed position or partially deployed position) into the stowed position for delivery or removal. Generally, the catheter 1100 may have the same or substantially similar features as the catheters 300, 500. Accordingly, certain details of the catheter 1100 may not be further described in detail below to avoid repetition and/or obscuring more pertinent details of the embodiment. Briefly, with reference to FIG. 11A, the catheter 1100 includes an elongate member 1102, an expandable member 1104 coupled thereto, and a flexible substrate 1106 coupled to the expandable member 1104 in a similar fashion and with similar functionality as described previously with respect to catheters 300, 500.

With reference to FIG. 11A, the catheter 1100 further includes an adjustment member 1108 that is coupled to one or both of the flexible substrate 1106 and the expandable member 1104, wherein the adjustment member 1108 is adjustable to retain the flexible substrate 1106 in the stowed position against the expandable member 1104. In some embodiments, the adjustment member 1108 is further adjustable to drive the flexible substrate 1106 from the deployed position or an expanded position back to the stowed position for removal of the catheter 1100. Additional details of the adjustment member 1108 and its functionality are described below.

FIG. 11A illustrates one example embodiment where the adjustment member 1108 is coupled directly to the flexible substrate 1106. The adjustment member 1108 may comprise any suitable member that facilitates application of tension against the flexible substrate 1106 and allows control of the tension as needed via adjustments of the adjustment member 1108. For example, in one embodiment, the adjustment member 1108 may be tension element, such as a cable or a line that may be formed of one or more fibers (e.g., braided fibers, suture line, Kevlar fibers, etc.). In this embodiment, the tension element is coupled to the flexible substrate 1106 and extends proximally along the length of the elongate member 1102 to a proximal end 1114 of the elongate member 1102. Tension may be applied to the tension element to tighten or retain the flexible substrate 1106 in the stowed position. Tension may be released from the tension element to allow the flexible substrate 1106 to transition into the deployed or expanded position.

With reference to FIG. 11A, the flexible substrate 1106 includes a base 1110 having one or more apertures 1112 formed thereon. In some embodiments, the apertures may include slits, eyelets, openings, or other suitable attachment or anchor points formed on the base 1110 of the flexible substrate 1106. The adjustment member 1108 may be coupled in any suitable manner to the flexible substrate 1106 via at least some of the one or more apertures 1112. For example, in one embodiment, the adjustment member 1108 may be weaved through at least some of the one or more apertures 1112 to couple the adjustment member 1108 to the flexible substrate 1106.

As noted previously, the adjustment member 1108 is configured to retain the flexible substrate 1106 in the stowed configuration against the expandable member 1104. In one example, tension may be applied on the adjustment member 1108, such as by pulling the adjustment member 1108 outwardly (or proximally) along the proximal end 1114 of the elongate member 1102, to tighten the adjustment member 1108 against the one or more apertures 1112 and retain the flexible substrate 1106 in a compact, stowed configuration. In some embodiments, the tension applied to the adjustment member 1108 may adjust the flexible substrate 1106 and draw the flexible substrate 1106 into a tighter, rolled configuration against the expandable member 1104. The adjustment member 1108 may be tightened on demand by an operator to reduce overall profile of the catheter 1100 such as during key workflow steps as needed. In some embodiments, the adjustment member 1108 may be coupled to the robotically-assisted manipulator system 100 (or other suitable system) to provide actuation, control, and/or electrical power to the flexible substrate 1106 as needed. Control via the manipulator system may facilitate fine adjustments to provide tighter control of the flexible substrate 1106 as needed. The adjustment member 1108 may be coupled to the flexible substrate 1106 in advance of delivery to the treatment site or on demand at the treatment site and prior to treatment. In embodiments in which the catheter 1100 is actuated by a robotically-assisted manipulator system (e.g., manipulator assembly 102), the catheter 1100 may include or be coupled to a drive unit (e.g., drive unit 204) having one or more drive inputs that removably couple to and receive power from drive elements, such as actuators, of the manipulator assembly as described above. The drive inputs of the catheter 1100 may be coupled to the adjustment member 1108 to actuate the adjustment member 1108. For example, in some embodiments in which the adjustment member 1108 includes a tension element such as cables, the cables may be wrapped around one or more capstans coupled to the drive inputs such that when the capstans are rotated in a first direction (e.g., via actuation of drive outputs of the manipulator system), the capstans cause the cables to pay in to tighten the flexible substrate 1106. When the capstans are rotated in a second direction opposite the first direction, the capstans may cause the cables to pay out to loosen the flexible substrate 1106 and permit the flexible substrate 1106 to expand (e.g., via expansion of the expandable member 1004).

In some embodiments, the adjustment member 1108 is further adjustable to control movement of the flexible substrate 1106 from the stowed position to the deployed position after an initial adjustment to retain the flexible substrate 1106 in the stowed position. For example, the adjustment member 1108 may be initially tightened to retain the flexible substrate 1106 in a compact, stowed configuration as described above. Thereafter, the adjustment member 1108 may be released to release tension against the flexible substrate 1106 and facilitate movement of the flexible substrate 1106 from the stowed position to the deployed position, such as in response to radial expansion of the expandable member 1104 in a similar fashion as described previously. In some embodiments, the adjustment member 1108 may thereafter be tensioned or otherwise tightened after deployment to guide or drive the flexible substrate 1106 from the deployed configuration back to the stowed configuration to prepare the catheter 1100 for removal.

In other embodiments, tension of the adjustment member 1108 may be released by cutting the adjustment member 1108 to facilitate adjustment of the flexible substrate 1106. For example, the adjustment member 1108 may be designed to form a continuous loop that may be cut adjacent the proximal portion 1114 of the elongate member 1102 to release the tension against the flexible substrate 1106. In other embodiments, the adjustment member 1108 may be cut adjacent the flexible substrate 1106, such as via a cutting instrument (not shown) that may be introduced in any suitable manner, such as through a working channel of the endoscope. After the adjustment member 1108 is cut, it may be removed from the flexible substrate 1106 to facilitate desired expansion of the flexible substrate 1106.

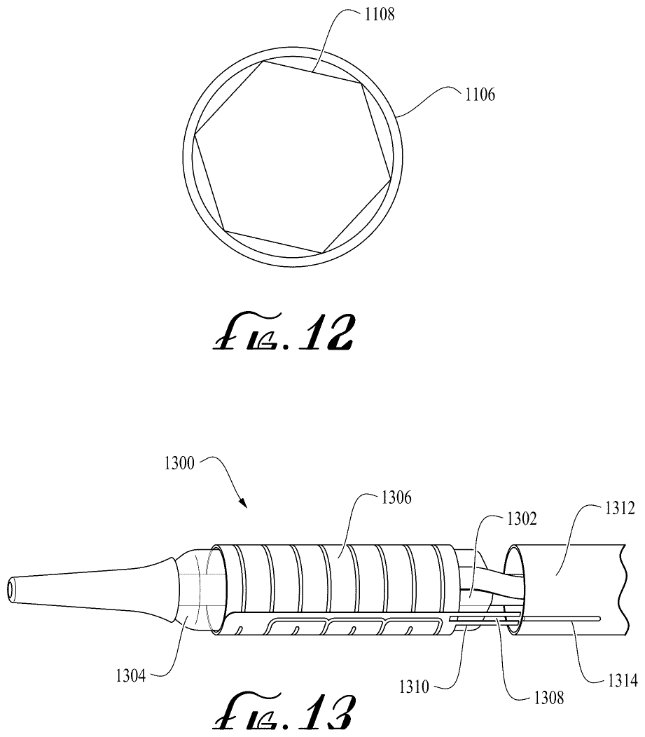

In some embodiments, the one or more apertures 1112 may be arranged in specific patterns (such as the zig-zag pattern shown in FIG. 11A) to provide additional control characteristics of the flexible substrate 1106. For example, with collective reference to FIGS. 11A and 12, the adjustment member 1108 may be weaved or otherwise coupled to the one or more apertures 1112, where the adjustment member 1108 contacts the flexible substrate 1106 at certain radial positions (see a schematic end view in FIG. 12) to restrict or limit expansion of the flexible substrate 1106 beyond a predetermined outer diameter. When the adjustment member 1108 is held in position and/or tightened, the flexible substrate 1106 is locked to expanding to a maximum diameter that is equal to the predetermined outer diameter. Once the predetermined outer diameter has been reached, the adjustment member 1108 restricts further expansion beyond that point. In some embodiments, the target diameter may be adjusted as needed by tightening or loosening tension on the adjustment member 1108. Restricting the target diameter to a predetermined threshold may help ensure that the flexible substrate 1106 expands as much as needed for effective tissue treatment, but prevents overexpansion of the flexible substrate 1106 to help avoid potential issues with retraction of the flexible substrate 1106 for removal upon completion of the treatment. In some embodiments, after the flexible substrate 1106 is deployed and treatment is completed, the adjustment member 1108 may be tightened to control retraction of the flexible substrate 1106 and drive the flexible substrate 1106 from the expanded, deployed position back to a compact, stowed position against the expandable member 1104 to facilitate catheter removal.

In the above examples, the adjustment member 1108 is coupled to the flexible substrate 1106 and operable to retain and/or otherwise control the flexible substrate 1106 relative to the expandable member 1104. In other embodiments, as noted previously, the adjustment member 1108 may alternatively, or additionally, be coupled to the expandable member 1104, where the adjustment member 1108 adjusts the expandable member 1104 to retain the flexible substrate 1106 in the stowed position against the expandable member 1104 and/or otherwise accomplish the same or similar objectives as described above. For example, in some embodiments, an adjustment member 1108 is coupled to the expandable member 1104 and the adjustment member 1108 is configured to move the expandable member 1104 from an expanded position into the stowed position. FIGS. 11B-11C depict example embodiments where adjustment members 1108 in the form of tension elements are coupled to expandable members 1104, which may be inflatable balloon members.

FIG. 11B depicts a schematic cross section of a tension element or adjustment member 1108 coupled to an expandable member 1104. In FIG. 11B, the adjustment member 1108 is coupled to a channel 1103 of the expandable member 1104, wherein the channel 1103 is separate from an expansion chamber 1101 of the expandable member 1104. The adjustment member 1108 may be coupled to the channel 1103 by an adhesive, a bonding agent, a press fit connection, a fastener, and the like. In some embodiments, the adjustment member 1108 is coupled to the channel 1103 by being embedded to the channel 1103 during molding. The channel 1103 may be integrally formed with the expandable member 1104 or may be attached to the expandable member 1104 (e.g., by an adhesive, a bonding agent, fasteners, or the like). The expansion chamber 1101 is configured to receive a fluid (such as air or another gas, a liquid such as saline, etc.) for expanding the expandable member 1104 and to release the fluid from the expansion chamber 1101 to deflate the expandable member 1104. When the fluid is removed from the expansion chamber 1101, tension can be applied to the adjustment member 1108 to move the expandable member 1104 into the stowed position and/or tension can be applied to the adjustment member 1104 to retain the expandable member 1104 in the stowed position. The flexible substrate (not shown) surrounds and is coupled to the expandable member 1104 such that the flexible substrate remains tightly wrapped when the expandable member 1104 is retained in the stowed position. Tension can be released from the adjustment member 1108 to allow the expansion chamber 1101 to expand when fluid is received in the expansion chamber 1101 and also thereby expand the flexible substrate. In FIG. 11B, the channel 1103 for the adjustment member 1108 extends to outside an outer expanded diameter of the expandable member 1104. FIG. 11C depicts another schematic cross section of a tension element adjustment member 1108 coupled to an expandable member 1104. In FIG. 11C, the channel 1103′ for the adjustment member 1108 is embedded in a wall of the expandable member 1104. For example, the channel 1103′ is recessed relative to an outer expanded diameter of the expandable member 1104. The channel 1103′ may be formed and the adjustment member 1108 may be embedded into the channel 1103′ during molding of the expandable member 1104.

FIG. 11D illustrates a flexible substrate 1120 in accordance with another example embodiment. To avoid obscuring more pertinent aspects of the embodiment, FIG. 11D schematically illustrates a standalone flexible substrate 1120 but it should be understood that the flexible substrate 1120 may be coupled to an expandable member (not shown) for use with a delivery catheter in a similar fashion as described in prior embodiments (such as FIGS. 5, 8-10, and 11A). Accordingly, the features described herein relating to the flexible substrate 1120 may be combined with catheters described in prior embodiments or other similar medical instruments. With reference to FIG. 11D, the flexible substrate 1120 includes an adjustment member 1122 coupled thereto and configured for retaining the flexible substrate 1120 in a rolled, stowed configuration against the expandable member. As illustrated, when the flexible substrate 1120 is in the rolled configuration, various layers of the flexible substrate 1120 overlap or stack on one another. For example, in one example configuration, the flexible substrate 1120 may include a first layer 1124 (e.g., an outermost layer in FIG. 11D), a second layer 1126 underneath the first layer 1124, a third layer 1128 underneath the second layer 1126, and so on. It should be understood that the flexible substrate 1120 may include any suitable number of layers that may depend on various factors, such as an overall length of the flexible substrate 1120 and/or an expansion diameter of the expandable member.

In one embodiment, the adjustment member 1122 is configured for coupling two or more layers of the flexible substrate 1120. For example, the adjustment member 1122 may couple the first layer 1124 and the second layer 1126 together. When coupled to the layers 1124, 1126, the adjustment member 1122 resists the unwrapping or unraveling of the flexible substrate 1120 that may otherwise occur when the flexible substrate 1120 encounters resistance or other forces acting on the first layer 1124 during catheter delivery.

In one example, the adjustment member 1122 may have a generally C-shaped profile including an elongate body segment 1130 having a first coupling portion 1132 and a second coupling portion 1134 disposed on respective end portions of the elongate body segment 1130. In one configuration, the elongate body segment 1130 is positioned such that it extends in an axial direction of the flexible substrate 1120 and abuts against an interior portion of the layers being coupled. For example, the elongate body segment 1130 may be positioned abutting an innermost or bottommost layer being coupled (e.g., the second layer 1126 in FIG. 11D) as illustrated. The coupling portions 1132, 1134 may each include a first segment 1136, 1138 extending from the elongate body segment 1130 and a second segment 1140, 1142 extending from the respective first segments 1136, 1138. In one configuration, the first segments 1136, 1138 may be generally orthogonal to the elongate body segment 1130 and the second segments 1140, 1142 may be generally orthogonal to the first segments 1136, 1138 (and generally parallel with the elongate body segment 1130). As illustrated in FIG. 11D, the first segments 1136, 1138 serve to brace the end portions of the captured layers 1124, 1126 of the flexible substrate 1120 to restrain lateral movement of the flexible substrate 1120 in the axial direction and the second segments 1140, 1142 help prevent unwrapping or unraveling of the flexible substrate 1120 in a radial direction. In some embodiments, the adjustment member 1122 may be adhered to or otherwise coupled to the outermost layer (e.g., the first layer 1126) of the flexible substrate 1120 to ensure the adjustment member 1122 remains in place against the flexible substrate 1120. For example, the second segments 1140, 1142 of the adjustment member 1122 may be adhered to the first layer 1126 of the flexible substrate 1120 (e.g., via an epoxy, adhesive, mechanical fasteners, or the like). In alternate arrangements, the elongate body segment 1130 may be positioned abutting the outermost layer of the flexible substrate 1120 being coupled (e.g., the first layer 1124) with the first coupling portion 1132 and the second coupling portion 1134 adhered or otherwise coupled to the innermost layer being coupled (e.g., the second layer 1126).

In one example embodiment, the adjustment member 1122 may be modified to essentially replace the continuous, elongate body segment 1130 with two smaller segments (not shown) such that the adjustment member 1122 includes a plurality of spaced apart, individual adjustment members, for example in the shape of two C-shaped hooks positioned on opposite ends of the flexible substrate (e.g., one on a proximal end of the flexible substrate 1120 and one on a distal end of the flexible substrate 1120). In this arrangement, the first coupling portion 1132 includes segments 1136, 1140 and a first elongate body segment (e.g., a proximal portion of segment 1130). Similarly, the second coupling portion 1134 includes segments 1138, 1142 and a second elongate body segment (e.g., a distal portion of segment 1130), with the first elongate body segment being spaced apart from the second elongate body segment.

Further referring to FIG. 11D, in some embodiments where the elongate body segment 1130 is to be positioned between the layers of the rolled flexible substrate 1120, the adjustment member 1122 is coupled to the flexible substrate 1120 by inserting the adjustment member 1122 between the layers of the rolled flexible substrate 1120 such that the elongate body segment 1130 abuts a lower layer of the flexible substrate 1120 to be captured and with the first coupling portion 1132 and the second coupling portion 1134 extending beyond the end portions of the lower layer of the flexible substrate 1120. Next, the first segments 1136, 1138 are bent away from the elongate body segment 1130 and towards an outer layer of the flexible substrate 1120 to be captured by the adjustment member 1122. Next, the second segments 1140, 1142 are bent into contact with the outer layer of the flexible substrate 1120 and bonded to the flexible substrate. In alternate embodiments, the adjustment member 1122 is coupled to the flexible substrate 1120 prior to placing the flexible substrate 1120 in the rolled configuration. For example, the second coupling portions 1132, 1134 of the adjustment member 1122 may be first bonded to portions of a flat flexible substrate 1120 that form the outermost layer when rolled. Then during rolling of the flexible substrate 1120, the elongate body segment 1130 may be positioned between the rolled layers. In embodiments where the elongate body segment 1130 is to be positioned abutting the outermost layer of the flexible substrate 1120, similar assembly steps occur but with the coupling portions 1132, 1134 being bonded to the inner layer of the flexible substrate 1120.

FIG. 11E illustrates another example of an adjustment member 1150 configured for retaining a flexible substrate 1152 in a similar fashion as described above with reference to FIG. 11D. In the illustrated example, the adjustment member 1150 is designed such that it forms a continuous loop around the captured layers of the flexible substrate 1152. The adjustment member 1150 includes a first elongate segment 1154, a second elongate segment 1156, and a third segment 1158 and a fourth segment 1160 spanning between opposing ends of the elongate segments 1154, 1156. With reference to FIG. 11E, in one example configuration, the second elongate segment 1156 extends in the axial direction of the flexible substrate 1152 and abuts against an interior portion of the bottommost captured layer of the flexible substrate 1152 (e.g., the second layer 1162). Similarly, the first elongate segment 1154 extends in the axial direction and abuts against the outermost layer of the flexible substrate 1152 (e.g., a first layer 1164). Together with the third and fourth segments 1158, 1160, the elongate segments 1154, 1156 restrain the flexible substrate 1152 in a rolled, stowed configuration against the expansion member (not shown) in a similar fashion as described above with reference to FIG. 11D. In some examples, the adjustment member 1150 may be adhered to or otherwise coupled to the outermost captured layer (e.g., the first layer 1164) of the flexible substrate 1152. In other examples, the adjustment member 1150 may not be adhered or directly coupled to any layer of the flexible substrate 1152.

As noted above, FIGS. 11D and 11E provide example embodiments for retaining the flexible substrate 1120, 1152 in a rolled, stowed configuration against the expandable member (not shown), where an adjustment member 1122, 1150 helps resist the unwrapping or unraveling of the flexible substrate 1120, 1152 in response to resistance or other forces acting on the flexible substrate 1120, 1152 during catheter delivery. In an alternate embodiment, the flexible substrate may instead include one or more segments extending from a peripheral side portion thereof in a similar fashion as segment 516 of the flexible substrate 502 of FIG. 5. In this configuration, the one or more segments of the flexible substrate may be tucked underneath the rolled flexible substrate and coupled (such as by an adhesive or in another suitable fashion) against the outermost layer of the flexible substrate (e.g., in a similar fashion as segments 1140, 1142 of FIG. 11D) along the proximal and/or distal portions of the flexible substrate to retain the flexible substrate in a stowed configuration.

FIG. 11F illustrates another example of a flexible substrate 1180 including one or more adjustment members 1194 (illustrated as arm segments) along edge portions of the flexible substrate 1180 for retaining the flexible substrate 1180 in a rolled, stowed configuration against an expandable member in accordance with examples described herein. With reference to FIG. 11F, the flexible substrate 1180 is substantially similar to the flexible substrates described in previous embodiments. For example, the flexible substrate 1180 includes a base 1182 that may be comprised of an electrically insulating polymer material, such as polyimide, polyester, or other suitable thermoplastic or thermosetting polymer film. The base 1182 includes a header portion (e.g., distal portion) 1184, an opposite footer portion (e.g., proximal portion) 1186, a first peripheral side portion 1188, and a second peripheral side portion 1190. The base 1182 further includes an electrode array 1192 disposed thereon. As noted above and illustrated in FIG. 11F, the flexible substrate 1180 includes a plurality of adjustment members 1194 extending outwardly from the header and footer portions 1184, 1186. In one example embodiment, the adjustment members 1194 may range in length from approximately 5-15 mm.

In one embodiment, the adjustment members 1194 may be thermally treated to create a hook shape for the adjustment members 1194 (i.e., the adjustment members 1194 are treated to provide a thermal memory of a hook shape). When the flexible substrate 1180 is rolled into its stowed configuration, the hooked adjustment members 1194 are arranged into position along the proximal and distal portions of the rolled flexible substrate 1180. In this position, the hooked adjustment members 1194 retain the flexible substrate 1180 in the rolled, stowed configuration in a similar fashion as the C-shaped brackets described above with reference to FIGS. 11D and 11E.

It should be understood that in other example embodiments, the flexible substrates 1120, 1152, 1180 may have any number of layers, and the adjustment members 1122, 1150, 1194 may be arranged to capture any suitable number of layers of the flexible substrates 1120, 1152, 1180. For example, in the illustrated embodiments, the adjustment members 1122, 1150, 1194 encapsulate the outermost two layers of the flexible substrates 1120, 1152, 1180. In other embodiments, however, the adjustment members 1122, 1150, 1194 may instead capture three or more layers of the flexible substrates 1120, 1152, 1180 as desired. In addition, in some embodiments, a plurality of adjustment members 1122, 1150 and/or 1194 may be provided. The plurality of adjustment members 1122, 1150 and/or 1194 may be at the same or different circumferential portions of the rolled flexible substrate 1120, 1152, 1180, and may capture the same layers or different layers of the flexible substrate 1120, 1152, 1180.

FIG. 13 illustrates a catheter 1300 with keyed mating features designed for facilitating retraction of the flexible substrate 1306 after deployment to aid in removing the catheter 1300 from the treatment site in accordance with an example embodiment. Generally, the catheter 1300 may have the same or substantially similar features of any of the catheters 300, 500. Accordingly, certain details of the catheter 1300 may not be further described in detail below to avoid repetition and/or obscuring more pertinent details of the embodiment. Briefly, with reference to FIG. 13, the catheter 1300 includes an elongate member 1302, an expandable member 1304 coupled thereto, and a flexible substrate 1306 coupled to the expandable member 1304 in a similar fashion and with similar functionality as described previously with respect to catheters 300, 500.

In some embodiments, the flexible substrate 1306 retains its low-profile, stowed configuration against the expandable member 1304 via its internal spring force. However, once the flexible substrate 1306 is expanded, it tends to lose some of its retention force. In some embodiments, after various expansion/retraction cycles, the flexible substrate 1306 may not be rolled as compactly against the expandable member 1304 when compressed into the stowed position. In addition, in some embodiments, the expandable member 1304 may not fully deflate back to its initial state once the fluid pressure is removed after treatment. Accordingly, the catheter 1300 may lose the original low-profile configuration post-deployment as compared to the pre-deployment profile, which may increase the difficulty in removing the catheter 1300 with minimal or no complications. Accordingly, the catheter 1300 is designed with features to help resolve these issues to facilitate catheter removal.

With reference to FIG. 13, the flexible substrate 1306 may include one or more mating features 1308 formed thereon. For example, in one embodiment, the flexible substrate 1306 may include the one or more mating features 1308 formed on an extended tail region 1310. The one or more mating features 1308 may include any suitable feature operable to engage a corresponding member. In one embodiment, the mating features 1308 may include an embossed region, such as a notch or a slot, or a raised region, such as a post or a ridge, or other suitable element. The catheter 1300 may further include a sheath 1312 having one or more reciprocal mating features 1314 with corresponding elements designed to mate with the one or more mating features 1308 on the flexible substrate 1306. In an example operation, after deployment of the flexible substrate 1306, the sheath 1312 may be advanced toward the flexible substrate 1306 to align the corresponding mating features 1308, 1314 with one another. Once the corresponding features 1308, 1314 on the flexible substrate 1306 and the sheath 1312 are aligned and mated, the flexible substrate 1306 may be pulled proximally and retracted into the sheath 1312. In some embodiments, the sheath 1312 may also be used to apply torque on the flexible substrate 1306 to drive it toward the stowed configuration within the sheath 1312 for retraction after treatment is complete. In embodiments in which the catheter 1300 is actuated by a robotically-assisted manipulator system, the mating features 1308 for articulating the flexible substrate 1306 may be part of the drive inputs of the drive unit at the proximal end portion of the catheter 1300. The mating features 1308 may be operatively coupled to reciprocal drive outputs of a manipulator assembly to place and retain the flexible substrate 1306 in the stowed configuration (e.g., by rotating the drive outputs of the manipulator assembly in a first direction). Optionally, the flexible substrate 1306 may be returned to the deployed configuration by rotating the manipulator drive outputs in a second direction opposite to drive the mating features 1308 in the second direction either alone or in combination with expanding the expandable member 1304.

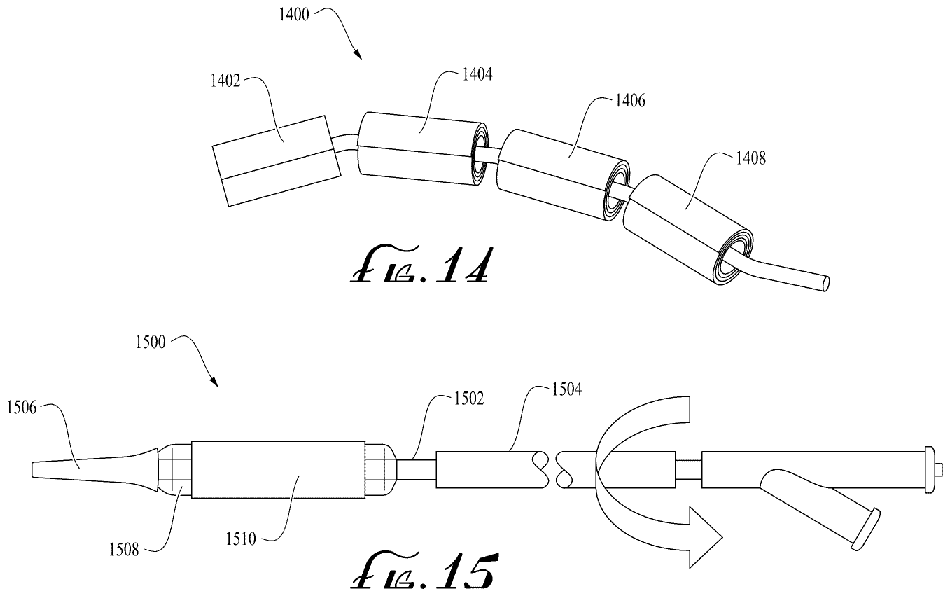

FIG. 14 is a schematic illustration of a segmented flexible substrate 1400 for use with any of the catheter designs described herein in accordance with an example embodiment. With reference to FIG. 14, the flexible substrate 1400 may be divided into multiple segments 1402, 1404, 1406, 1408 as illustrated. In some embodiments, the segments 1402, 1404, 1406, 1408 may each include wiring (not shown) to independently power the electrode arrays (not shown) supported thereon relative to one another. In other embodiments, the segments 1402, 1404, 1406, 1408 may be electrically coupled with one another and work in tandem as essentially a multi-segmented electrode array. In either configuration, the segmented flexible substrate 1400 may provide additional flexibility for an endoscopically delivered catheter to aid in navigating through tortuous anatomy. In addition, this design may also increase the overall region of tissue that may be simultaneously treatable, thereby reducing overall treatment time and minimizing potential tissue damage that may be caused when repositioning the catheter for subsequent treatments. In some embodiments, the segments 1402, 1404, 1406, 1408 may be wrapped around a single expandable member (not shown) operable to substantially simultaneously expand each segment 1402, 1404, 1406, 1408 against the tissue for treatment. In other embodiments, each segment 1402, 1404, 1406, 1408 may instead have an independently controllable expandable member for selectively expanding the corresponding segment 1402, 1404, 1406, 1408 as needed.

FIG. 15 is a schematic illustration of a catheter 1500 in accordance with one embodiment. With reference to FIG. 15, the catheter 1500 may include an inner elongate member 1502 (e.g., an inner shaft), an outer elongate member 1504 (e.g., an outer shaft), a distal tip 1506, an expandable member 1508, and a flexible substrate 1510 arranged in a similar fashion as described previously. In some embodiments, the inner elongate member 1502 and the outer elongate member 1504 may be made from a single piece of nitinol (nickel titanium) hypotube. This hypotube may be selectively laser cut to gain unique stiffness, push, track, and torque properties. The hypotube may help provide stiffness to resist kinking issues of the expandable member 1508, particularly when the expandable member 1508 is an inflatable balloon. In some embodiments, the distal tip 1506 may also be designed as an integral component together with the elongate members 1502, 1504 and may have a very closely pitched laser cut pattern to ensure high flexibility and facilitate insertion and navigation through the patient's anatomy.

In some embodiments, as the catheter 1500 is delivered into the esophagus and advanced toward a treatment site, the flexible substrate 1510 may tend to unfurl from the expandable member 1508 due to frictional and other forces applied thereon. As such, one approach during delivery is to torque the catheter 1500 counter-clockwise (e.g., in the direction of tightening) to help avoid potential unfurling of the flexible substrate 1510 and retain the flexible substrate 1510 in position against the expandable member 1508. This approach is not particularly ergonomic and will likely require multiple and ongoing twisting of the catheter 1500 during delivery to counteract the unfurling. Accordingly, in some embodiments, the catheter 1500 may further include a spring-based mechanism (not shown) for constantly applying torque against the catheter 1500 in the counter-clockwise direction as the catheter 1500 is advanced through the esophagus and to the treatment site. In a manual approach, the spring-based mechanism may be activated using a button, lever, or other suitable actuatable device on the catheter 1500 or in operable communication therewith. In some embodiments, the spring-based mechanism may be operated via a semi- or fully-robotic system (e.g., system 100 of FIG. 2) to mitigate any ergonomic issues for the operator. In some embodiments, this torquing motion may aid trackability of the catheter 1500 by reducing static friction associated therewith.

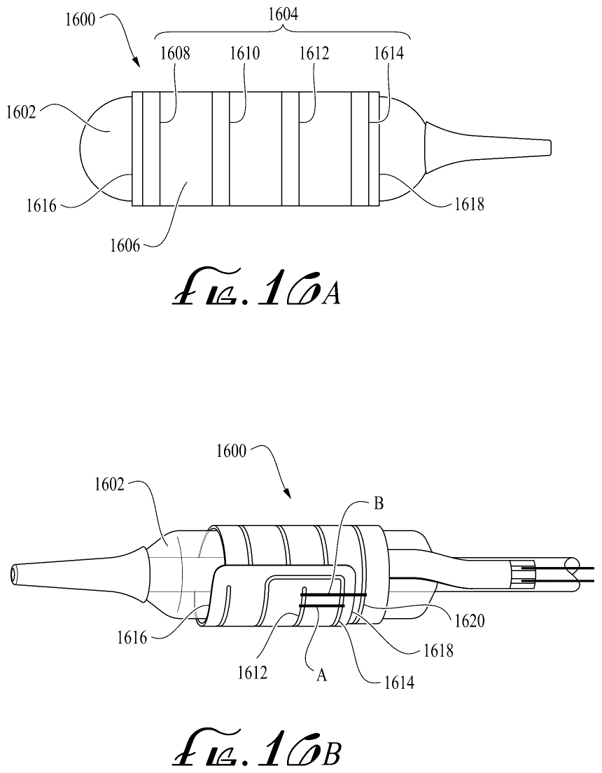

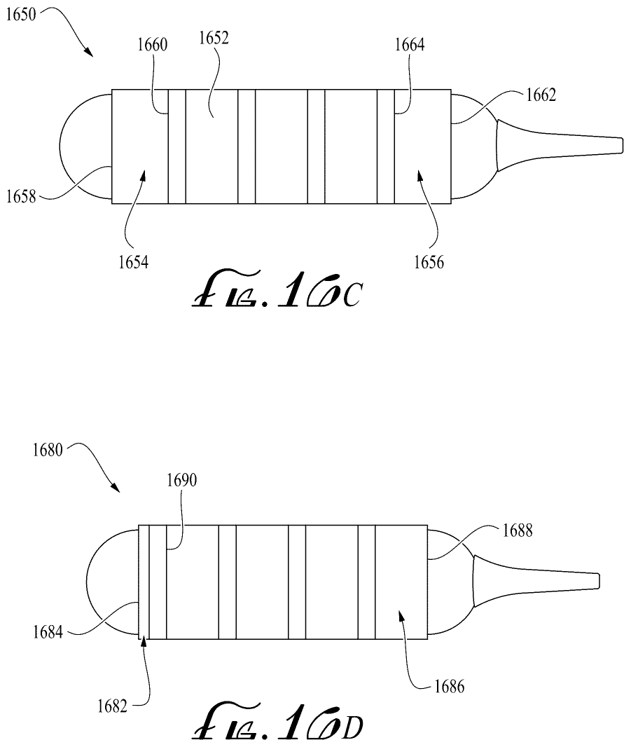

FIGS. 16A-16D collectively illustrate various examples of a flexible substrate and electrode array coupled to an expandable member for use with a catheter for treatment of target tissues in a similar fashion as described previously in prior example embodiments. Certain details of the catheter are not further described with reference to FIGS. 16A-16D to avoid repetition and/or obscuring more pertinent details of the illustrated embodiments. It should be understood that the description associated with other catheter embodiments described herein (e.g., catheters 300, 500, 800) may be incorporated with the additional details described with reference to FIGS. 16A-16D. With general reference to FIGS. 16A-16D, the following description provides various examples of flexible substrate configurations designed to help ensure desired performance of the electrode array and avoid creating unintended current paths between electrodes when the electrode array is deployed and energized for treatment.

FIG. 16A schematically illustrates a distal portion of a catheter (not shown) including a flexible substrate 1600 coupled to an expandable member 1602 in a similar fashion as described previously with reference to any of the catheter embodiments described herein. With reference to FIG. 16A, the flexible substrate 1600 includes an electrode array 1604 disposed along a base 1606 of the flexible substrate 1600. As described previously, the electrode array 1604 may take any suitable configuration operable for generating an electric field to provide electroporation and/or electrolysis for tissue treatment. For example, the electrode array 1604 may comprise a plurality of metal traces 1608, 1610, 1612, 1614 deposited on the flexible substrate 1600, where the metal traces 1608, 1610, 1612, 1614 include any suitable electrically conductive material, such as gold, copper, stainless steel, titanium, graphene, graphite, and the like. For use with electroporation in combination with electrolysis, the material of the traces 1608, 1610, 1612, 1614 may be selected such that the electrode material does not actively participate in the electrolysis products when current is applied and/or leave material residue. For example, the electrode material may be chosen to minimize transferring ions from the electrode material to the target tissue. For example, steel may be less preferred. Titanium and gold may have some degree of participation in the electrolysis process, however they may not generate a toxic residue and may be used in some examples. In some examples, titanium may be used and may be preferred. In some examples, stainless steel may be used. In some examples, two separate metals such as zinc and aluminum may be used. In one example arrangement, the traces 1608, 1610, 1612, 1614 are spaced apart from one another along the base 1606 to help ensure a desired current path when the electrode array 1604 is energized. For example, with reference to FIG. 16A, the traces 1608, 1610, 1612, 1614 are each 1-2 mm wide and may be separated from one another by a spacing of approximately 3 mm (or between 1-5 mm). The first trace (e.g., trace 1608) closest to a proximal edge portion 1616 of the base 1606 and the last trace (e.g., trace 1614) closest to a distal edge portion 1618 of the base 1606 are each offset from the respective edge portions 1616, 1618 by a buffer of approximately 1 mm. In other words, there is a 1 mm spacing between the proximal and distal portions 1616, 1618 of the base 1606 and the position of the respective traces 1608, 1614.

In some examples, a small buffer region along the proximal and/or distal portions 1616, 1618 of the base 1606 may result in the inadvertent exposure of underlying trace segments (e.g., trace segments lying in layers underneath the outermost portion of the wrapped flexible substrate 1600) of the electrode array 1604 during expansion of the expandable member 1602 and/or during positioning of the catheter for treatment. The inadvertent exposure of underlying trace segments may lead to imprecise energy delivery during treatment, to noise in impedance measurements, to the creation of unintended current paths between traces of the electrode array 1604, and/or to other unwanted performance behaviors or characteristics of the flexible substrate 1600. For example, FIG. 16B illustrates one example of the flexible substrate 1600 in a partially shifted position after expansion of the expandable member 1602.

With reference to FIG. 16B, in some examples, layers of the flexible substrate 1600 may unintentionally shift as described above. In FIG. 16B, the flexible substrate 1600 is illustrated in a shifted position where the proximal and distal portions 1616, 1618 of the flexible substrate 1600 are each shifted or offset proximally relative to an original position (e.g., the position illustrated in FIG. 16A) of the flexible substrate 1600. This proximal shift of the flexible substrate 1600 uncovers and exposes an underlying trace segment 1620 of the electrode array 1604 adjacent the trace 1614. As illustrated in FIG. 16B, the exposed trace segment 1620 is adjacent the trace 1614 and disrupts a normal current path (illustrated as path A) between adjacent traces 1612, 1614 and creates an unintended current path (illustrated as path B) between traces 1612, 1614 and the exposed trace segment 1620 when the electrode array 1604 is energized. To help minimize the impact of positional shifts of the flexible substrate 1600, additional buffer regions along the proximal and/or distal portions 1616, 1618 of the base 1606 may be incorporated into the design of the flexible substrate 1600 as further described below.

FIG. 16C illustrates an example embodiment of a flexible substrate 1650 having a base 1652 with an extended proximal buffer region 1654 and an extended distal buffer region 1656. The proximal buffer region 1654 is disposed between a proximal portion 1658 of the base 1652 and a first trace 1660 and the distal buffer region 1656 is positioned between a distal portion 1662 and a second trace 1664. With reference to FIG. 16C, the additional buffer regions 1654, 1656 help ensure that trace segments (e.g., trace segment 1620 of FIG. 16B) in underlying wrapped portions of the flexible substrate 1650 are not exposed when the flexible substrate 1650 shifts either proximally or distally. In other words, the buffer regions 1654, 1656 of the base 1652 help ensure the underlying trace segments remain covered when the flexible substrate 1650 shifts to avoid inadvertent interaction between trace segments, such as those discussed with reference to FIG. 16B. In some embodiments, the buffer regions 1654, 1656 may each range from between 3 mm to 7 mm to provide a wide coverage range for any shifts of the flexible substrate 1650. In some embodiments, the buffer regions may be greater than or equal to a spacing between adjacent traces of the electrode array.

In some embodiments, such as the embodiment of FIG. 16C, the buffer regions may be symmetrical (e.g., have the same width) along the proximal and distal portions of the flexible substrate. In other embodiments, the flexible substrate may have a larger buffer region along the distal portion as compared to the proximal portion (or vice versa). For example, FIG. 16D illustrates an example of a flexible substrate 1680 with an asymmetric buffer design including a shorter buffer region 1682 along a proximal portion 1684 and a larger buffer region 1686 along a distal portion 1688. In some embodiments, the flexible substrate 1680 may tend to shift proximally during catheter delivery. For example, in some cases, the catheter may be advanced (e.g., moved forwardly in a proximal-to-distal direction) from a first treatment region to a second treatment region. As the catheter is advanced, corresponding reaction forces exerted on the flexible substrate 1680 push the outermost layer of the flexible substrate 1680 proximally (e.g., opposite the direction of catheter advancement), thereby resulting in the potential exposure of underlying trace segments along the distal portion 1688. Accordingly, since exposure along the distal portion 1688 of the flexible substrate 1680 is more likely as compared to exposure along the proximal portion 1684 when the catheter is advanced, a larger distal buffer region 1686 is advantageous.

Moreover, having a shorter proximal buffer region 1682 on the proximal portion 1684 of the flexible substrate 1680 may also be beneficial in certain embodiments. In some examples, an endoscope (or other medical instrument) may follow behind the catheter during delivery for visualization within the tissue lumen. In these examples, a larger proximal buffer region 1682 along the proximal portion 1684 of the flexible substrate 1680 may hinder visibility of the traces (e.g., trace 1690) of the electrode array from the endoscope since the flexible substrate 1680 is opaque. Accordingly, having a shorter proximal buffer region 1682 may help improve visibility of the electrode array on the flexible substrate 1680, while the larger distal buffer region 1686 helps minimize potential exposure of underlying trace segments as described above.

In other examples, the flexible substrate (not shown) may have the opposite configuration of the flexible substrate 1680 of FIG. 16D, where the flexible substrate includes a larger buffer region along the proximal portion and a shorter buffer region along the distal portion. This configuration may help minimize exposure of underlying traces along the proximal portion of the flexible substrate such as may occur when the catheter is moved proximally (e.g., in an opposite fashion as described with reference to FIG. 16C). For example, in some cases, the catheter may be retracted (e.g., moved rearwardly in a distal-to-proximal direction) from a first treatment region to a second treatment region. As the catheter is retracted, corresponding reaction forces exerted on the flexible substrate push the outermost layer of the flexible substrate distally, thereby resulting in the potential exposure of underlying trace segments along the proximal portion. Accordingly, the larger buffer region on the proximal portion is beneficial in these cases.

It should be understood that example embodiments provided herein of both the design of catheter delivery systems and the potential clinical applications associated therewith are not intended to be limiting. Many other configurations of delivery systems exist, as well as applications that would benefit from the use of the disclosed subject matter. In addition, it is to be appreciated that any one of the above embodiments or processes, or specific features associated therewith, may be combined with one or more other embodiments and/or processes or be separated and/or performed amongst separate devices or device portions in accordance with the present systems, devices, and methods.

Finally, the disclosure is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

1. A system for tissue treatment, the system comprising:
an elongate member;
an expandable member coupled to the elongate member, wherein the expandable member is radially expandable relative to the elongate member; and
a flexible substrate including an electrode array disposed thereon, the flexible substrate having a header portion and a footer portion, and a first peripheral side portion and a second peripheral side portion each spanning between the header portion and the footer portion, the flexible substrate including a first arm segment extending outwardly from the header portion and a second arm segment extending outwardly from the footer portion, wherein the flexible substrate surrounds at least a portion of the expandable member when in a stowed position with one of the first arm segment or the second arm segment of the flexible substrate coupled to the expandable member to retain the flexible substrate against the expandable member in the stowed position, and wherein the flexible substrate is expandable from the stowed position to a deployed position in response to radial expansion of the expandable member.

2. The system of claim 1, wherein the one of the first arm segment or the second arm segment is coupled to the expandable member when the flexible substrate is in the deployed position.

3. The system of claim 1, further comprising one or more electrical wires coupled to the electrode array and configured to power the electrode array, wherein the one or more electrical wires retain the flexible substrate in position along a proximal portion of the elongate member.

4. The system of claim 1, wherein the elongate member includes a distal portion and a proximal portion, and wherein the expandable member is coupled to the elongate member between the distal portion and the proximal portion.

5. The system of claim 4, wherein the one of the first arm segment or the second arm segment of the flexible substrate is coupled to the expandable member adjacent the distal portion of the elongate member to retain the flexible substrate against the expandable member.

6. The system of claim 5, wherein the other of the first arm segment or the second arm segment of the flexible substrate is coupled to one or more electrical wires configured to power the electrode array, the one or more electrical wires retaining the flexible substrate against the expandable member adjacent the proximal portion of the elongate member.

7. The system of claim 1, the flexible substrate further including a leading edge defined along one of the header portion or the footer portion of the flexible substrate corresponding with the one of the first arm segment or the second arm segment coupling the flexible substrate to the expandable member when the flexible substrate is in the stowed position, the system further including a first cushion adjacent the leading edge of the flexible substrate to protect against tissue trauma during system delivery.

8. The system of claim 7, the flexible substrate further including a trailing edge defined along the other of the header portion or the footer portion of the flexible substrate, the system further including a second cushion adjacent the trailing edge of the flexible substrate to protect against tissue trauma during system removal.

9. The system of claim 1, the flexible substrate further including a leading edge defined along one of the header portion or the footer portion of the flexible substrate corresponding with the one of the first arm segment or the second arm segment coupling the flexible substrate to the expandable member when the flexible substrate is in the stowed position, wherein the expandable member is partially expanded to form a first cushion adjacent the leading edge of the flexible substrate to protect against tissue trauma during system delivery.

10. The system of claim 9, the flexible substrate further including a trailing edge defined along the other of the header portion or the footer portion of the flexible substrate, wherein the expandable member is partially expanded to form a second cushion adjacent the trailing edge of the flexible substrate to protect against tissue trauma during system removal.

11. The system of claim 1, wherein the first arm segment and the second arm segment are aligned relative to one another along a first axis extending across the flexible substrate from the header portion to the footer portion.

12. The system of claim 1, wherein the first arm segment and the second arm segment are aligned relative to one another along one of the first peripheral side portion or the second peripheral side portion of the flexible substrate.

13. The system of claim 1, wherein the first arm segment is substantially orthogonal to the header portion and the second arm segment is substantially orthogonal to the footer portion.

14. The system of claim 1, wherein the other of the first arm segment or the second arm segment is free of the electrode array.

15. The system of claim 1, further comprising one or more apertures formed on the flexible substrate, wherein the one or more apertures are disposed between portions of the electrode array.

16. The system of claim 1, the flexible substrate further including a top surface and an opposite bottom surface, wherein the first peripheral side portion of the flexible substrate includes a first edge surface spanning from the top surface to the bottom surface, and wherein the second peripheral side portion of the flexible substrate includes a second edge surface spanning from the top surface to the bottom surface, the system further comprising a protective coating layer covering at least one of the first edge surface or the second edge surface of the flexible substrate.

17. The system of claim 16, wherein the electrode array is disposed on the top surface of the flexible substrate, and wherein the protective coating layer covers at least a portion of the electrode array.

18. The system of claim 1, the flexible substrate further including a top surface and an opposite bottom surface, the system further comprising a protective coating layer covering at least a portion of the top surface of the flexible substrate.

19. The system of claim 1, wherein the electrode array includes a plurality of spaced apart traces with a first trace being closest to a proximal end of the flexible substrate and a second trace being closest to a distal end of the flexible substrate, and wherein the flexible substrate includes a buffer region without an electrode trace, the buffer region being between at least one of: a) a distance between the first trace and the proximal end of the flexible substrate; or b) a distance between the second trace and the distal end of the flexible substrate.

20. The system of claim 19, wherein the buffer region is between both a) the distance between the first trace and the proximal end of the flexible substrate; and b) the distance between the second trace and the distal end of the flexible substrate.
